(19) United States
(12) Patent Application Publication
(10) Pub. No.: US 2026/0218109 A1
(43) Pub. Date: Jul. 30, 2026

(54) CULTURING DEVICE AND METHOD

(71) Applicant: METHANE MITIGATION VENTURES LIMITED, NELSON (NZ)

(72) Inventors: Stephen John Sowerby, Dunedin (NZ); Peter Francis Fennessy, Dunedin (NZ); Martin David Jones, Port Chalmers (NZ)

(21) Appl. No.: 19/158,211

(22) PCT Filed: Feb. 13, 2024
(86) PCT No.: PCT/NZ2024/050013

§ 371 Date: Aug. 20, 2025

(30) Foreign Application Priority Data
Feb. 20, 2023 (NZ) 797376

(51) Int. Cl. C12M 1/34; C12M 3/06

(52) U.S. Cl. CPC C12M 41/32; C12M 27/16; C12M 41/12; C12M 41/40

(57) ABSTRACT

The present invention is concerned with an in vitro culturing device and methods for culturing microorganisms derived from a mammalian gut, and in particular microorganisms derived from the gut of ruminant and pseudo-ruminant animals. The culturing devices according to the present invention comprise a vessel for containing a culture, at least one conduit fluidically connected to said vessel providing a path for the culture, a pressure adjustment means, at last one sensor configured to detect at least one physical or chemical property of the culture, a volatile fatty acid adsorption means disposed inside said conduit, and a control module, wherein the control module is configured to move the culture and thereby cause at least a portion of said culture to move between said vessel and said conduit and change at least one parameter of said culture. Accordingly, the culturing devices according to the present invention have been designed to accurately reflect some important aspects of gut organ function by mimicking natural processes of gut organ physiology such as the mixing and pressure dynamics of fluids and solids within a gut organ, as well as a pressure adjustment means that can sense pressure variations and permit the escape of fermentation gases aligned with natural foregut eructation or hindgut flatulence observed in (e.g.) ruminant animals.

### TECHNICAL FIELD

The present invention relates to a culturing device, and to the use of a culturing device for culturing microorganisms from an animal gut, for example microorganisms from a ruminant animal foregut.

### BACKGROUND OF THE INVENTION

Culturing microorganisms from an animal gut, particularly the ruminant animal foregut, to discover, develop, and assess feeds, treatments, additives, supplements, and devices, to improve the health and productivity and/or reduce methane emissions from farmed animals is complicated by the complexity of animal biology, the diversity of gut microorganisms and the structural features of the gastrointestinal environment.

Live animals are useful to discover, develop, and assess, feeds, treatments, additives supplements, and devices. However, the breeding, housing, and maintenance of animals is costly and risky. The administration of tests, taking of measurements, and the extraction of samples from animals is difficult, which may cause discomfort and stress and may compromise measurements. Additionally, natural differences between animals introduce variance, and the scale of testing required to achieve statistical significance makes the use of live animal testing time-consuming, expensive and undesirable.

A culturing device is required to culture microorganisms from an animal gut that more accurately reflects gut organ physiology and conveniently provides measurements, analytical samples, and testing. Such a device can be scaled, is easy to manage and provides a useful alternative to live animal testing for the discovery, development, and assessment of feeds, treatments, additives, supplements, and devices.

The present invention provides devices and methods to culture microorganisms from an animal gut to discover, develop, and assess feeds, treatments, additives, supplements, and devices.

### SUMMARY OF THE INVENTION

The inventions described and claimed herein have many attributes and embodiments including, but not limited to, those set forth or described or referenced in this Summary of the Invention. It is not intended to be all inclusive and the inventions described and claimed herein are not limited to or by the features or embodiments identified in this Summary of the Invention, which is included for purposes of illustration only and not restriction.

In an aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means; and
(v) a control module,
wherein the at least one sensor is configured to detect at least one physical or chemical property of the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a temperature adjustment means; and
(vi) a control module,
wherein the at least one sensor is configured to detect at least one physical or chemical property of the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means; and
(v) a control module,
wherein the at least one sensor is configured to detect at least one physical or chemical property of the culture, and wherein the sensor comprises a dissolved hydrogen sensor (dH₂) and the dH₂ sensor is configured to measure an amount or concentration of dissolved hydrogen in the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a temperature adjustment means; and
(vi) a control module,
wherein the at least one sensor is configured to detect at least one physical or chemical property of the culture, and wherein the sensor comprises a dissolved hydrogen sensor (dH₂) and the dH₂ sensor is configured to measure an amount or concentration of dissolved hydrogen in the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a volatile fatty acid adsorption means; and
(vi) a control module,
wherein the at least one sensor is configured to detect at least one physical or chemical property of the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a temperature adjustment means;
(vi) a volatile fatty acid adsorption means; and
(vii) a control module,
wherein at the least one sensor is configured to detect at least one physical or chemical property of the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a volatile fatty acid adsorption means; and
(vi) a control module,
wherein the at least one sensor is configured to detect at least one physical or chemical property of the culture, and wherein the sensor comprises a dissolved hydrogen sensor (dH₂) and the dH₂ sensor is configured to measure an amount or concentration of dissolved hydrogen in the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a temperature adjustment means;
(vi) a volatile fatty acid adsorption means; and
(vii) a control module,
wherein at the least one sensor is configured to detect at least one physical or chemical property of the culture, and wherein the sensor comprises a dissolved hydrogen sensor (dH₂) and the dH₂ sensor is configured to measure an amount or concentration of dissolved hydrogen in the culture, and wherein said control module is configured to move said vessel and thereby cause at least a portion of said culture to move between said vessel and said conduit and change at least one parameter of said culture.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a control module;
wherein the at least one sensor is configured to detect at least one physical or chemical property of the culture, and wherein said control module (1) is configured to move said vessel and thereby cause at least a portion of said culture to move between said vessel and said conduit and change at least one parameter of said culture and/or (2) is configured to control at least one parameter of the culturing device in response to the physical or chemical property of the culture or in response to an external signal.

In another aspect of the present invention there is provided a culturing device comprising:
(i) at least one vessel for containing a culture;
(ii) at least one sensor;
(iii) at least one conduit fluidically connected to said vessel providing a path for said culture;
(iv) a pressure adjustment means;
(v) a temperature adjustment means;
(vi) a control module,
wherein the at least one sensor is configured to detect at least one physical or chemical property of the culture, and wherein said control module (1) is configured to move said vessel and thereby cause at least a portion of said culture to move between said vessel and said conduit and change at least one parameter of said culture and/or (2) is configured to control at least one parameter of the culturing device in response to the physical or chemical property of the culture or in response to an external signal.

In another aspect of the present invention there is provided a method for controlling a culturing device comprising:
(i) inoculating a culturing device as described herein with a culture; and
(ii) activating the control module to control at least one parameter of the culturing device in response to a change in a physical or chemical property of the culture as detected by the sensor; or
(iii) activating the control module to control at least one parameter of the culturing device in response to an external instruction,
thereby controlling the culturing device.

In another aspect of the present invention there is provided a method for adjusting at least one parameter of a culture or culturing device comprising:
(i) inoculating a culturing device as described herein with a culture; and
(ii) adjusting at least one parameter of the culture or culturing device using an input signal to activate the control module.

In another aspect of the present invention there is provided a method for determining a physical or chemical change in a culture comprising:
(i) inoculating a culturing device as described herein with a culture;
(ii) measuring a physical or chemical property of the culture at an initial time point;
(iii) measuring the physical or chemical property of the culture according to (ii) at a later time point; and
(iv) determining any change in the physical or chemical property of the culture between the initial time point (ii) and the later time point (iii).

In another aspect of the present invention there is provided a method for evaluating the effect of an intervention on a culture comprising:
(i) inoculating a culturing device as described herein with a culture;
(ii) introducing said intervention to the culturing device; and
(iii) measuring at least one parameter associated with the culture from the at least one sensor,
thereby evaluating the effect of the intervention on the culture.

In another aspect of the present invention there is provided a method for evaluating the effect of feed on a culture comprising:
(i) inoculating a culturing device as described herein with a culture;
(ii) introducing the feed to the culture device; and
(iii) measuring at least one parameter associated with the culture from the at least one sensor,
thereby evaluating the effect of the feed on the culture.

In another aspect of the present invention there is provided a method for evaluating the effect of a feed supplement on a culture comprising:
(i) inoculating a culturing device as described herein with a culture;
(ii) introducing the feed supplement to the culture device; and
(iii) measuring at least one parameter associated with the culture from the at least one sensor,
thereby evaluating the effect of the feed supplement on the culture.

In another aspect of the present invention there is provided a method for evaluating the effect of a medicament on a culture comprising:
(i) inoculating a culturing device as described herein with a culture;
(ii) introducing the medicament to the culture device; and
(iii) measuring at least one parameter associated with the culture from the at least one sensor,
thereby evaluating the effect of the medicament on the culture.

In another aspect of the present invention there is provided a method for evaluating the effect of a microorganism on a culture comprising:
(i) inoculating a culturing device as described herein with a culture;
(ii) introducing the microorganism to the culture device; and
(iii) measuring at least one parameter associated with the culture from the at least one sensor,
thereby evaluating the effect of the microorganism on the culture.

In another aspect of the present invention there is provided a method for evaluating the effect of a microorganism inhibitor on a culture comprising:
(i) inoculating a culturing device as described herein with a culture;
(ii) introducing the microorganism inhibitor to the culture device; and
(iii) measuring at least one parameter associated with the culture from the at least one sensor,
thereby evaluating the effect of the microorganism inhibitor on the culture.

In another aspect of the present invention there is provided a method for evaluating the effect of a microorganism enhancer on a culture comprising:
(i) inoculating a culturing device as described herein with a culture;
(ii) introducing the microorganism enhancer to the culturing device; and
(iii) measuring at least one parameter associated with the culture from the at least one sensor,
thereby evaluating the effect of the microorganism enhancer on the culture.

In another aspect of the present invention there is provided a method for evaluating the effect of a methanogen inhibitor on a culture comprising:
(i) inoculating a culturing device as described herein with a culture;
(ii) introducing the methanogen inhibitor to the culturing device; and
(iii) measuring the amount of methane produced by the culture,
thereby evaluating the effect of the methanogen inhibitor on the culture.

In another aspect of the present invention there is provided a method for evaluating the effect of a methanogenesis inhibitor on a culture comprising:
(i) inoculating a culturing device as described herein with a culture;
(ii) introducing the methanogenesis inhibitor to the culturing device; and
(iii) measuring the amount of methane produced by the culture,
thereby evaluating the effect of the methanogenesis inhibitor on the culture.

In another aspect of the present invention there is provided a method for determining the volatile fatty acid (VFA) potential associated with a material comprising:
(i) inoculating a culturing device as described herein with a culture;
(ii) determining a reference VFA concentration produced by the culture;
(iii) introducing the material into the culture; and
(iv) measuring the VFA concentration produced by the culture following introduction of the material (iii);
wherein, the VFA potential associated with the material is calculated by subtracting the VFA concentration determined at (ii) from the VFA concentration determined at (iv).

In another aspect of the present invention there is provided a method for reducing the volatile fatty acid (VFA) concentration in a culture comprising:
(i) inoculating a culturing device as described herein with a culture;
(ii) inserting a VFA adsorption means to said culturing device;
(iii) waiting an interval of time;
wherein said concentration of VFA is reduced when VFA is adsorbed from said culture on the said VFA adsorption means.

In another aspect of the present invention there is provided a method for measuring the volatile fatty acid (VFA) concentration in a culture comprising:
(i) inoculating a culturing device as described herein with a culture;
(ii) inserting a VFA adsorption means to said culturing device;
(iii) waiting an interval of time;
(iv) extracting said VFA adsorption means from said culturing device;
(v) eluting adsorbed VFA from said VFA adsorption means; and,
(vi) reporting at least one measure of VFA,
wherein said concentration of VFA is determined when said reporting is evaluated.

In another aspect of the present invention there is provided a method for determining the methane potential associated with a material comprising:
(i) inoculating a culturing device as described herein with a culture;
(ii) determining a reference methane concentration produced by the culture;
(iii) introducing a material into the culture; and
(iv) measuring the methane concentration produced by the culture following introduction of the material (iii);
wherein, the methane concentration derived from the material is calculated by subtracting the methane concentration determined at (ii) from the methane concentration determined at (iv).

In another aspect of the present invention there is provided a method for determining the rate of methane production associated with a material comprising:
(i) inoculating a culturing device as described herein with a culture;
(ii) determining a reference methane concentration produced by the culture at a first time point;
(iii) introducing a material into the culture; and
(iv) measuring the methane concentration produced by the culture following introduction of the material (iii) at a second time point;
wherein, the rate of methane production associated with the material is calculated by subtracting the methane concentration determined at (ii) from the methane concentration determined at (iv) as a function of time.

### BRIEF DESCRIPTION OF THE FIGURES

FIG. 1 depicts an example of a culturing device according to the present invention.

FIG. 2 depicts an example of culturing device variations in a culturing device according to the present invention.

FIG. 3 depicts an example of a culturing device according to the present invention in a predetermined position.

FIG. 4 depicts an example of a culturing device according to the present invention in a predetermined position for administration of a material.

FIG. 5 depicts an example of a culturing device according to the present invention in a predetermined position for extraction of a material.

FIG. 6 depicts an example of a vessel of a culturing device according to the present invention at different stages of operation.

FIG. 7 depicts an example of a manufactured vessel and a VFA adsorption means of a culturing device according to the present invention.

FIG. 8 shows an example of a manufactured culturing device according to the present invention.

FIG. 9 shows an example recording of prescribed movement of a culturing device according to the present invention.

FIG. 10 shows an example recording of prescribed movement and the changing of pressure in a vessel of a culturing device according to the present invention.

FIG. 11 shows an example of data from microorganism feeding and administration of methanogenesis inhibitor to a culturing device according to the present invention.

FIG. 12 shows an example recording of prescribed movement and the changing of pressure in a vessel of a culturing device according to the present invention.

### DETAILED DESCRIPTION

### General Definitions

Unless specifically defined otherwise, all technical and scientific terms used herein shall be taken to have the same meaning as commonly understood by one of ordinary skill in the art to which the inventions belong (for example, in immunology, immunohistochemistry, protein chemistry, and biochemistry).

The term “and/or”, e.g., “X and/or Y” shall be understood to mean either “X and Y” or “X or Y” and shall be taken to provide explicit support for both meanings or for either meaning.

The term “a” or “an” refers to one or more than one of the entity specified; for example, “a pressure adjustment means” or may refer to one or more pressure adjustment means. As such, the terms “a” or “an”, “one or more” and “at least one” can be used interchangeably herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally equivalent products, compositions and methods are clearly within the scope of the invention, as described herein.

Any example or embodiment described herein shall be taken to apply mutatis mutandis to any other example or embodiment unless specifically stated otherwise.

### Selected Definitions

The term “gut organ” as used herein includes, but is not limited to, the foregut, hindgut, oesophagus, rumen, reticulum, pseudo-rumen, reticulo-rumen, omasum, abomasum, stomach, true stomach, caecum, small intestine, large intestine, bowel, and colon.

The term “rumen” should be understood as meaning the foregut organs of ruminant and pseudo-ruminant animals, where the fermentation of consumed feed predominantly occurs. Specifically, “rumen” may refer to the first and largest stomach chamber, or alternatively a combination of the first and second stomach chambers in the alimentary canal of animals from the suborder Ruminantia (ruminants) including wild and domesticated animals, for example cattle, goats, sheep, bison, buffalo, yaks, deer. The term “rumen” may also refer to the first stomach chamber in the alimentary canal of animals from the suborder Tylopoda (pseudo-ruminants) including antelope, camels, alpacas, and llamas.

The term “gut content” as used herein should be understood as meaning the interior and contents of the gut organs of live or dead animals including but not limited to, a gut organ derived fluid, a gut organ derived solid, a gut organ derived semisolid, an oesophageal sample, a sample procured from a fistula, a rectal sample, an excretion sample, a vomit sample and a faecal sample. The gut content may further comprise: microorganisms, for example, archaea, bacteria, fungi, viruses and protozoa; organic and inorganic materials, for example, fluids, liquids and gases, dissolved gas, hydrogen (H), molecular hydrogen (H₂), ionic hydrogen (H⁺), oxygen (O), molecular oxygen (O₂), carbon dioxide (CO₂), nitrogen (N), molecular nitrogen (N₂), ammonia (NH₃), ammonium (NH₄⁺), hydrogen sulfide (H₂S), methane (CH₄), water, mineral salts, ions, bicarbonate, buffers; biomolecules, for example, amino acids, peptides, proteins, nucleic acids, carbohydrates, fatty acids, volatile fatty acids including but not limited to acetic acid, acetate, propionic acid, propionate, butyric acid, butyrate, isobutyric acid, isobutyrate, valeric acid, valerate, hexanoic acid, hexanoate, heptanoic acid and heptanoate, co-factors, flavin co-factors and co-factor F420.

The term “liquor” or “vessel liquor” should be understood as meaning to include all of the contents of the vessel disclosed herein and includes:
(i) all of the materials inoculated into the vessel, for example, gut contents and buffers;
(ii) all of the materials administered into the vessel including feeds, supplements, and treatments, for example forage, pasture, grass, Gramineae, legume, Leguminosae, clover, lucerne, fodder, preserved, straw, hay, silage, baleage, brassica, herb, chicory, plantain, grains, oats, barley, maize; and feed supplements, drug, inhibitor, methanogenesis inhibitor, organohalogen, haloform, chloroform, bromoform, iodoform, seaweed, Asparagopsis sp., Asparagopsis extract, Asparagopsis matter, polyphenols, alliin, iso-alliin, allicin, Mootral™, garlic extract, garlic matter, 3-Nitrooxypropanol (3-NOP), Bovaer™, device, bolus, electronic bolus; and
(iii) all of the materials generated in the vessel including all of the products of enteric fermentation, for example microorganisms selected from the group comprising archaea, methanogens, bacteria, mycobacterium, fungi, viruses, and protozoa; organic and inorganic material including fluids, liquids and gases, dissolved gas, atmospheric gas, purified or refined gases, and acidity regulators selected from the group comprising hydrogen (H), molecular hydrogen (H₂), ionic hydrogen (H⁺), oxygen (O), molecular oxygen (O₂), carbon dioxide (CO₂), nitrogen (N), molecular nitrogen (N₂), ammonia (NH₃), ammonium (NH₄⁺), hydrogen sulfide (H₂S), methane (CH₄), water, mineral salts, ions, bicarbonate, buffer; biochemicals, amino acids, peptides, proteins, nucleic acids, carbohydrates, fatty acids, volatile fatty acids including but not limited to acetic acid, acetate, propionic acid, propionate, butyric acid, butyrate, isobutyrate, isobutyric acid, valerate and valeric acid, co-factors, flavin co-factors, co-factor F420 and polyphenols.

As used herein, the term “methanogenesis” should be understood to mean methane production by methanogenic organisms that leads to methane release into the atmosphere. Example methanogenic organisms may be selected from the group consisting of: Archaea, ruminal archaea, the order Methanomicrobiales, the order Methanobacteriales, the order Methanosarcinales, the genus Methanobrevibacter, the genus Methanosphaera, the genus Methanomicrobium, the genus Methanobacterium, and the genus Methanosarcina.

### DETAILED DESCRIPTION

The gut organs of different animals contain diverse cultures of microorganisms including archaea, bacteria, fungi, viruses, and protozoa, that ferment a variety of feed material and produce a fermentation profile of various molecules. The fermentation profile typically comprises volatile fatty acids (VFAs, also referred to as short chain fatty acids (SCFA)), and includes predominantly acetate, propionate, butyrate, isobutyrate, and to a lesser extent valerate, isovalerate, and caproate, all of which are molecules used by animals as a form of energy for maintenance and growth. The fermentation profile also includes gas molecules, which predominantly includes but without limitation carbon dioxide (CO₂), molecular hydrogen (H₂), hydrogen sulfide (H₂S), and methane (CH₄). Methane is produced by methanogenic archaea (methanogens) by a process of methanogenesis in which hydrogen is used to reduce carbon dioxide (CO₂) to methane (CH₄). Although many animals produce methane in their gut organs, ruminant and pseudo-ruminant animals produce substantial quantities of methane in their foregut, which is emitted from the animal into the atmosphere by eructation. While emitted methane represents feed energy that is lost from the animal for maintenance and growth, it also poses an environmental problem as a potent greenhouse gas that traps heat in the atmosphere and contributes to climate change. The large numbers of ruminant and pseudo-ruminant animals farmed in modern civilisations means substantial methane emissions leading to undesirable losses in animal productivity coupled to undesirable greenhouse gas production. There is a widely recognised need to reduce methane emissions from farmed ruminant animals to improve animal productivity and reduce greenhouse gas production.

The present invention, in broad terms, relates to devices and methods used for culturing microorganisms derived from the gut organs of animals to discover, develop, and assess feeds, treatments, supplements, and devices to improve animal productivity and/or reduce greenhouse gas production.

Importantly, the devices according to the present invention have been conceived and developed to accurately mimic some key aspects of gut organs creating a model system for evaluation of feeds, treatments, supplements, and devices etc that does not require complex infrastructure and/or considerable expense.

The measurement of (e.g.) fermented gas or energy potentials associated with animal feeds, feed supplements, microorganism, microorganism enhancers etc which has been described for existing prior art solutions has relied either (a) on complex/expensive laboratory testing involving animals taken from their natural habitats or (b) bioreactors which are designed to accommodate one or more classes of microorganism but fail to accurately simulate gut organ physiology.

For example, measurement of methane production from a live animal can be achieved using a gas-tight respiration chamber that completely encloses the animal and captures emitted gases for analysis. However, such chambers are expensive to construct and to operate, and are limited to a small number of animals and permitted measurements.

Other approaches involve procuring samples for analysis via a canula surgically inserted in a living animal to provide direct access to gut cultures. Such installations incur surgical risks, require high levels of expertise to install, manage and use, and are expensive to establish and manage and have limited availability.

Similarly, while the development of bioreactors to culture gut organ microorganisms has previously been attempted, there are inherent limitations.

For example, mixing of cultures in previous bioreactors has relied on the use of paddles, impellors, stirrers, or plungers which directly contact and mix the culture in a stationary vessel, or alternatively, by swirling or uncontrolled mechanical agitation of a vessel to introduce circular or erratic motion to the culture. These methods of mixing establish large-scale centrifugal flow, or small-scale patterns of vortices, eddies, and turbulence, which compromises the natural stratification of the culture and limits the amount of fibrous material that can be introduced to the vessel and effectively mixed.

For example, the challenges of managing and mixing fibrous material has constrained previous approaches involving the culture of gut organ-derived microorganisms which methods have sought to strain the gut content from an animal to remove the fibrous material and obtain the pure liquor for inoculation, and previous bioreactors which actively limit or otherwise prevent fibre mat formation in the culture due to the predisposition of fibre to clump, adhere to bioreactor walls, and occlude tubes and pipes. However, the fibrous material and structure of the fibre mat of a ruminant or pseudo-ruminant animal contains microorganisms including at least important fungi known to play a critical role in plant matter decomposition which removal and prevention of fibre mat structure has a direct effect on the microorganism composition and the faithful performance of the in vitro culture.

Another example includes expensive gas management systems that are used to introduce CO₂ and nitrogen (N₂) gas treatments to flush the headspace of bioreactors and flush any administered materials free of oxygen (O₂) due to the prevailing belief that the gut content must be strictly anaerobic to permit methanogenesis.

However, these devices and methods are not sympathetic to the animal gut content and natural gut organ physiology, which (i) is repeatedly exposed to oxygen via the oesophagus due to the consumption of atmospheric gases during feeding, regurgitation and rumination, (ii) the in vivo mixing achieved by the slow contraction of the gut muscles to cause material tumbling and pressurisation of the gut content yet enable the composition and maintenance of a stratification of liquor, fibre mat, and headspace, (iii) the continued withdrawal of fermentation products by absorption of VFAs through gut epithelia, and (iv) the pressure-induced eructation of fermentation gases.

### Culturing Devices

The present invention seeks to address these limitations by providing a culturing device which more effectively mimics gut organ physiology. The devices according to the present invention achieve this outcome by providing the following novel features: (i) a conduit providing a path for the culture akin to the oesophagus of an animal through which air, feed, and treatments can be administered, instruments conveniently inserted and withdrawn, and samples easily extracted, (ii) adjustable and programmable positions, intervals of motion, and intervals of stillness applied to the movement of the vessel to enable the tumbling of its contents to accurately mimic the mixing and pressure dynamics of fluids and fibrous materials within a gut organ in vivo, (iii) a pressure adjustment means that can sense pressure variations and permit the escape of fermentation gases aligned with natural foregut eructation or hindgut flatulence observed in (e.g.) ruminant animals and (optionally) (iv) a cassette containing ion exchange media that, in one example, may be conveniently inserted and removed from the conduit, which contacts the liquor and removes volatile fatty acids from the culture in real time, which cassette can be conveniently exchanged. These features thereby mimic how gut cultures behave in a physiological sense as dietary material is processed for energy extraction which until now has not been achieved by in vitro culturing devices.

According to the culturing devices described herein, the novel features (i), (ii), (iii) and (iv) referred to immediately above may be present alone or in any combination within a device described herein including, but not limited to: (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); (i), (ii), (iii) and (iv).

The devices according to the present invention yield the production of methane gas at levels surprisingly comparable to an animal despite no active use of gas treatments to flush the headspace of the culturing vessel or the administered materials free of O₂. The devices according to the present invention further provide easily procured samples for analysis and dissolved hydrogen signals that reveal metabolic dynamics. The devices of the present invention are therefore a comparatively inexpensive option to live animal testing and provide superior performance over previously described bioreactors. Further still, the devices according to the present invention are adaptable to automation and can be easily scaled.

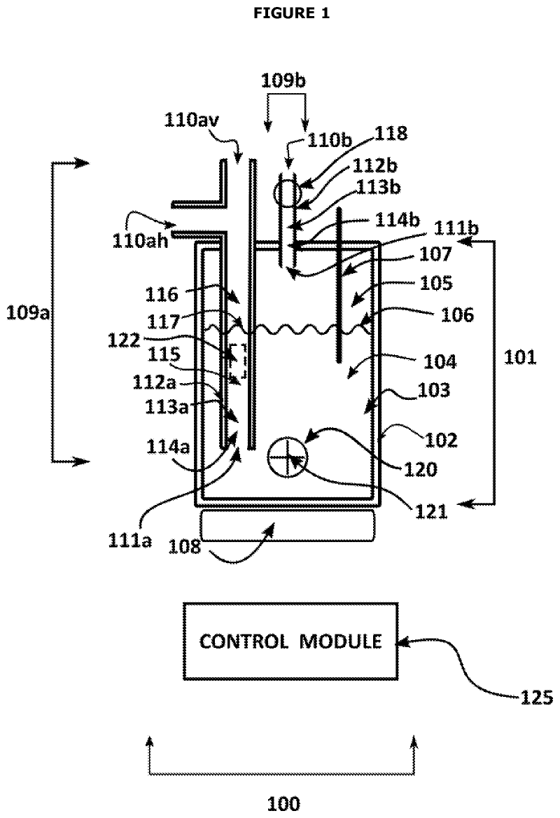

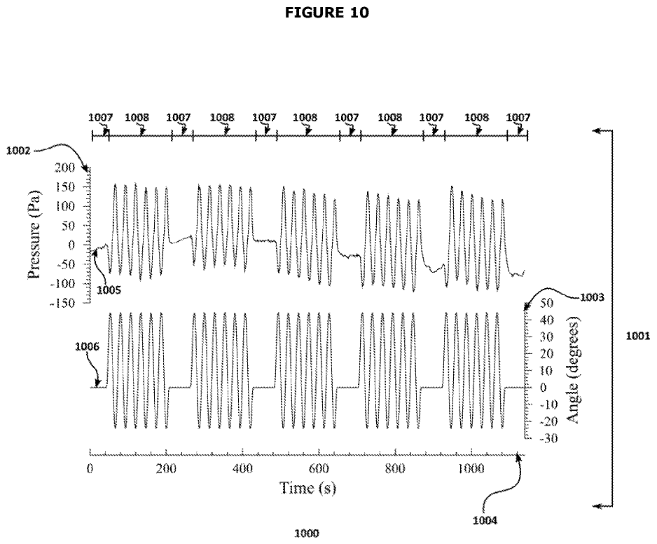

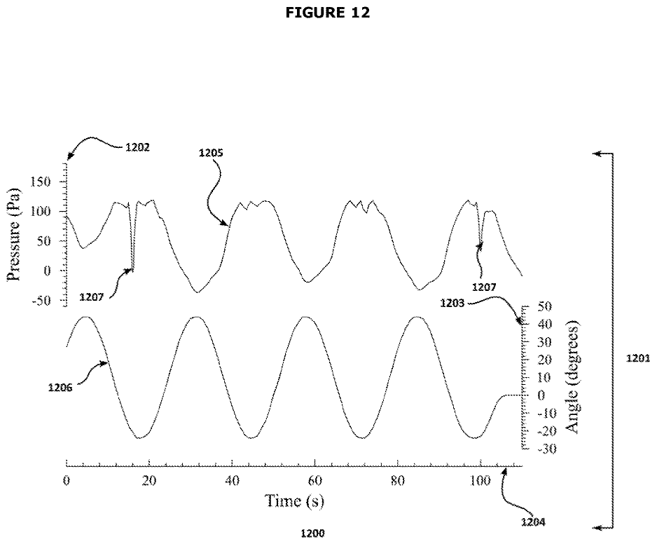

Exemplary devices according to the present invention developed and tested by the Applicant are presented in FIGS. 1-8, the structural and/or functional features for which are outlined in further detail below. Further, the data presented in FIGS. 10 and 12 provide an exemplary recording of prescribed movement and the changing of pressure in the vessel which accurately reflects the movement of liquor and gases in a gut organ.

Accordingly, in an aspect of the present invention there is provided a culturing device comprising:
(i) at least one vessel for containing a culture;
(ii) at least one sensor;
(iii) at least one conduit fluidically connected to said vessel providing a path for said culture;
(v) a pressure adjustment means;
(vi) a volatile fatty acid adsorption means disposed inside the conduit; and
(v) a control module,
wherein the at least one sensor is configured to detect at least one physical or chemical property of the culture, and wherein said control module is configured to move said vessel and thereby cause at least a portion of said culture to move between said vessel and said conduit and change at least one parameter of said culture.

In an example according to the devices of the present invention, the sensor comprises a dissolved hydrogen sensor (dH₂) and the dH₂ sensor is configured to measure an amount or concentration of dissolved hydrogen in the culture.

In another example according to the devices of the present invention, the at least one sensor is configured to directly contact said culture.

In another example according to the devices of the present invention, the control module is configured to move the vessel in a way which simulates the movement reflected in a gut organ, and in particular a gut organ derived from a ruminant animal.

In another example according to the devices of the present invention, the control module is configured to move the vessel in a way which simulates the movement of its contents by tumbling.

In another example according to the devices of the present invention, the pressure adjustment means is configured to generate fluctuations in pressure that would occur naturally in a gut organ (e.g. via eructation or flatulence), and in particular a gut organ derived from a ruminant animal.

In another example according to the devices of the present invention, the at least one parameter of said culture is selected from a change in pressure of said culture, the position of said culture, the level of said culture, the distribution of said culture, the volume of said culture, the composition of said culture and the volatile fatty acid (VFA) concentration of said culture.

In another example according to the devices of the present invention, the at least one parameter of said culture is a change in pressure.

In another example according to the devices of the present invention, said pressure adjustment means is selected from an airlock, a valve, a regulator, a pump, a vacuum, and a compressor.

In another example according to the devices of the present invention, the pressure adjustment means adjusts the pressure of the culture to atmospheric pressure, near atmospheric pressure, less than atmospheric pressure and greater than atmospheric pressure.

In another example according to the devices of the present invention, said VFA adsorption means is selected from a cationic solid, an anion exchanger, an anion exchange resin, a weak anion exchange resin, an ion exchange membrane, and a cassette comprising a cationic solid, an anion exchanger, an anion exchange resin, a weak anion exchange resin and/or an ion exchange membrane.

In another example according to the devices of the present invention, the VFA adsorption means adjusts the concentration of VFA in the culture to reduce the concentration of at least one VFA, to reduce the concentration of acetate, to reduce the concentration of propionate, to reduce the concentration of butyrate, to reduce the concentration of isobutyrate, to reduce the concentration of pentanoate, to reduce the concentration of valerate, to reduce the concentration of isovalerate, to reduce the concentration of hexanoate, to reduce the concentration of caproate and/or to reduce the concentration of heptanoate.

In another example according to the devices of the present invention, the culturing device further comprises a temperature adjustment means which is configured to adjust and/or maintain the vessel at a temperature value that approximates the temperature microenvironment of a gut organ.

In another example according to the devices of the present invention, said temperature adjustment means senses and reports the heat generated by the culture and adjusts the temperate of the culture to reduce the temperature of the culture or to increase the temperature of the culture.

In another example according to the devices of the present invention, said temperature adjustment means is selected from heating temperature adjustment means, a cooling temperature adjustment means, a maintained temperature adjustment means, a constant temperature adjustment means, an isothermal temperature adjustment means, a static temperature adjustment means, a dynamic temperature adjustment means, an incremental temperature adjustment means, a ramped temperature adjustment means, a stepped temperature adjustment means, a modulated temperature adjustment means, an active temperature adjustment means, a passive temperature adjustment means, an insulated temperature adjustment means, a thermally insulated temperature adjustment means, a vented temperature adjustment means, a jacketed temperature adjustment means, a water jacketed temperature adjustment means, an electronic temperature adjustment means, a heater element temperature adjustment means, a Peltier device, a heat sink, a fan, a pulse width modulation (PWM), all of which adjust the temperature to about 20° C. to about 25° C., about 30° C. to about 35° C., about 37° C., about 39° C., about 40° C. and about 45° C.

In another example according to the devices of the present invention, said vessel is selected from an open vessel, a closed vessel, a sealed vessel, a gas tight vessel, a transparent vessel, an opaque vessel, a formed vessel, a metal vessel, a polymer vessel, a glass vessel, a silicate vessel, a borosilicate vessel, a plastic vessel, a monolithic vessel, a composite material vessel, an assembled vessel, a 3D printed vessel, a cast vessel and a moulded vessel. In one example according to these and other aspects of the present invention, the vessel takes the form of a cassette, an insert, an exchangeable vessel, a replaceable vessel, a disposable vessel, and a recyclable vessel.

In yet another example according to the devices of the present invention, said portion of culture that moves between said vessel and said conduit is selected from about 1 percent, about 5 percent, about 10 percent, about 20 percent, about 30 percent, about 50 percent, about 80 percent, about 100 percent and includes without limitation the headspace, liquid, solid, semisolid, and effluent associated with said culture.

In yet another example according to the devices of the present invention, said control module moves said vessel to said first position for an interval of time and to at least one second position for an interval of time.

In yet another example according to the devices of the present invention, said first position is about ±1 degrees to about ±180 degrees from vertical. The term “about ±1 degrees to about ±180 degrees from vertical” is intended to mean about ±1, about ±2, about ±3, about ±4, about ±5, about #6, about ±7, about #8, about ±9, about #10, about #11, about ±12, about #13, about ±14, about ±15, about ±16, about #17, about ±18, about ±19, about ±20, about ±21, about ±22, about ±23, about ±24, about ±25, about ±26, about ±27, about ±28, about ±29, about ±30, about ±31, about ±32, about ±33, about ±34, about ±35, about ±36, about ±37, about ±38, about ±39, about ±40, about ±41, about ±42, about #43, about ±44, about ±45, about ±46, about ±47, about ±48, about ±49, about ±50, about ±51, about ±52, about ±53, about ±54, about ±55, about ±56, about ±57, about ±58, about ±59, about ±60, about ±61, about ±62, about ±63, about ±64, about ±65, about ±66, about ±67, about ±68, about ±69, about ±70, about ±71, about ±72, about ±73, about ±74, about ±75, about #76, about ±77, about ±78, about ±79, about #80, about ±81, about ±82, about ±83, about ±84, about ±85, about ±86, about ±87, about ±88, about ±89 and about ±90 and about #91, and about ±92, and about ±93, and about ±94, and about ±95, and about #96, and about ±97, and about ±98, and about ±99, and about ±100, and about ±101, and about ±102, and about ±103, and about ±104, and about ±105, and about ±106, and about ±107, and about ±108, and about ±109, and about ±110, and about ±111, and about ±112, and about ±113, and about ±114, and about ±115, and about ±116, and about #117, and about ±118, and about ±119, and about ±120, and about ±121, and about ±122, and about ±123, and about ±124, and about ±125, and about ±126, and about ±127, and about ±128, and about ±129, and about ±130, and about ±131, and about ±132, and about #133, and about ±134, and about #135, and about ±136, and about ±137, and about ±138, and about #139, and about ±140, and about ±141, and about ±142, and about ±143, and about ±144, and about #145, and about ±146, and about ±147, and about ±148, and about #148, and about ±150, and about ±151, and about ±152, and about ±153, and about ±154, and about #155, and about ±156, and about ±157, and about ±158, and about ±159, and about #160, and about ±161, and about ±162, and about ±163, and about #164, and about ±165, and about ±166, and about ±167, and about ±168, and about ±169, and about ±170, and about #171, and about ±172, and about ±173, and about ±174, and about ±175, and about ±176, and about ±177, and about ±178, and about ±179, and about #180 degrees from vertical.

In yet another example according the devices of the present invention, said second position is about ±1 degrees to about ±90 degrees from vertical.

In yet another example according to the devices of the present invention, said interval of time is selected from, about 1 second, about 30 seconds, about 1 minute, about 30 minutes, about 1 hour, about 2 hours, about 3 hours, about 6 hours, about 12 hours and about 1 day.

In yet another example according to the devices of the present invention, the conduit is selected from a hole, a port, an inlet, an outlet, a fluid inlet, a fluid outlet, a gas inlet, and a gas outlet.

In yet another example according to the devices of the present invention, the conduit is a rigid conduit, a flexible conduit.

In yet another example according to the devices of the present invention, the conduit is comprised of a hose, a pipe, a tube, a lumen, a drain, a self-sealing conduit, and a manifold.

In yet another example according to the devices of the present invention, the conduit is selected from a linear conduit, a branched conduit, a straight conduit, a S-bend conduit and a goose necked conduit.

In yet another example according to the devices of the present invention, at least one part of the conduit is located external to said vessel.

In yet another example according to the devices of the present invention, at least one part of the conduit is located internal to said vessel.

In yet another example according to the devices of the present invention, the conduit is selected from an open conduit, a closed conduit, a partially closed conduit, an occluded conduit, a partially occluded conduit, an adjustable conduit, a valved conduit, a gated conduit, a pumped conduit, and a pressurised conduit.

In yet another example according to the devices of the present invention, the conduit can incorporate a device to aid in the administration or extraction of materials contained in said vessel selected from a tube, an adsorbent, a resin, an ion exchange resin, a weak anion exchange resin, a strong anion exchange resin and a cation exchange resin, a cartridge, a cassette, an auger and a feed screw.

In yet another example according to the devices of the present invention, the conduit can incorporate a component to prepare and measure fermentation gases selected from a sensor, a filter, a tube, a scrubber, a gas scrubber, a H₂S scrubber and a H₂O trap.

In yet another example according to the devices of the present invention, the conduit is comprised of a metal, a polymer, a glass, a silicate, a borosilicate, a plastic, a monolith, and a composite material.

In yet another example according to the devices of the present invention, the sensor is selected from a synchronous (real-time) sensor, an asynchronous sensor, a sensor located external to vessel, a sensor located internal to vessel, a physical sensor, an electronic sensor, a chemical sensor, a biochemical sensor, an electrochemical sensor, an optical sensor, a mechanical sensor, a micro-electromechanical sensor (MEMS), an accelerometer, an inertial measurement unit (IMU), a gyroscope, a motion-tracking sensor, a rotational sensor, a positional sensor, an end-stop sensor, a temperature sensor, a mass sensor, a volume sensor, an acidity sensor, an electrochemical potential sensor, a pH sensor, a redox potential (Eh) sensor, an oxidation redox potential (ORP) sensor, a pressure sensor, a partial pressure sensor, a concentration sensor, a fluid sensor, a fluid displacement sensor, a gas sensor, a gas flow sensor, a dissolved gas sensor, a hydrogen (H) sensor, a molecular hydrogen (H₂) sensor, an ionic hydrogen (H⁺) sensor, a dissolved H₂ sensor, an oxygen (O) sensor, a molecular oxygen (O₂) sensor, a dissolved O₂ sensor, a carbon dioxide (CO₂) sensor, a dissolved CO₂ sensor, a nitrogen (N) sensor, a molecular nitrogen (N₂) sensor, a hydrogen sulfide (H₂S) sensor, a methane (CH₄) sensor, a VFA sensor, an acetate sensor, a propionate sensor, a butyrate sensor, an isobutyrate sensor, a pantoate sensor, a valerate sensor, an isovalerate sensor, a hexanoate sensor, a caproate sensor, a heptanoate sensor, a chromatograph, a liquid chromatograph a high performance liquid chromatograph, and a gas chromatograph.

In yet another example according to the devices of the present invention, the control module comprises a moving means wherein the moving means is selected from mechanical moving means, motorized moving means, robotic moving means, reciprocating moving means, pivot, axis moving means, axis of rotation moving means, axle moving means, uniaxial moving means, biaxial moving means, triaxial moving means, motor-driven moving means, electric moving means, alternating current (AC) moving means, direct current (DC) moving means, stepper moving means, servo moving means, direct-drive moving means, geared moving means, gear moving means, cam moving means, cam gear moving means, cam shaft moving means, pully moving means, belt pulley moving means, sprocket moving means, belt drive moving means, linkage moving means, controlled moving means, automated moving means, semi-automated moving means, motor controller moving means, pneumatic moving means, hydraulic moving means, device moving means, computer-driven moving means, networked computer-driven moving means, peripheral moving means, module moving means, sensor moving means, circuit moving means, electronic moving means, microprocessor moving means, memory moving means, communication interface moving means, bus moving means, Inter-integrated circuit (I2C) moving means, universal asynchronous receiver/transmitter (UART) moving means, serial bus moving means, universal serial bus (USB) moving means, synchronous serial connection (SPI) moving means, 1-Wire® moving means, ethernet moving means, wireless area network-based (WAN) moving means, WiFi moving means, Bluetooth moving means, network enabled moving means, instruction moving means, algorithm moving means, code moving means, routine moving means, subroutine moving means, firmware moving means, software moving means, error-detection moving means, checksum moving means, hash moving means, data moving means, data packet moving means, identification moving means, radio frequency identification (RFID) moving means, bar code moving means, QR code moving means.

The purpose of the control module according to the devices described herein is two-fold. The primary (and essential) function of the control module is to move the culture (e.g.) in a spatial orientation that most closely resembles the passage/fluidics of liquor and gas in a gut organ (note includes feed) and ensures that at least a portion of said culture moves between the vessel and the conduit and changes at least one parameter of the culture. For example, refer to FIG. 10 where angular movement (degrees) and pressure (Pa) associated with a culture that is cultured within a culturing device according to the present invention is measured as a function of time.

The secondary (and non-essential) function of the control module is to manipulate the culturing device that is either responsive to at least one physical or chemical property of the culture (i.e. mediated in/directly through the sensor) and/or is responsive to an external instruction. Accordingly, at least one property of the culture or the culturing device may be adjusted or changed by the control module to more accurately simulate physiological gut organ functions.

As such, the control module may be configured to detect an internal signal or an external instruction and activate an instruction responsive module to instruct an operation means to undertake a pre-determined function. For example, to introduce or remove liquor or gas from the culture through the conduit, or to adjust the pressure and/or the temperature of the vessel, or to remove a VFA or to introduce buffer both of which are mechanisms to control pH.

Accordingly, in another example according to the devices of the present invention the control module may further comprise:
(i) at least one instruction responsive module;
(ii) at least one operation means; and
(iii) optionally, at least one signal sensing module.

In other examples of the devices described herein, the instruction responsive module is configured to be responsive to an external instruction, which in turn may activate the operation means to undertake at least one operation in accordance with the teaching provided herein.

In other examples of the devices described herein, the signal sensing module is selected from a synchronous (real-time) signal sensing module, an asynchronous signal sensing module, a signal sensing module that is located external to vessel, a signal sensing module that is located internal to vessel, a physical signal sensing module, an electronic signal sensing module, a chemical signal sensing module, a biochemical signal sensing module, an electrochemical signal sensing module, an optical signal sensing module, a mechanical signal sensing module, a rotational signal sensing module, a positional signal sensing module, an end-stop signal sensing module, a temperature signal sensing module, a mass signal sensing module, a volume signal sensing module, an acidity signal sensing module, an electrochemical potential signal sensing module, a pH signal sensing module, a redox potential (Eh) signal sensing module, an oxidation redox potential (ORP) signal sensing module, a pressure signal sensing module, a partial pressure signal sensing module, a concentration signal sensing module, a fluid signal sensing module, a fluid displacement signal sensing module, a gas signal sensing module, a gas flow signal sensing module, a dissolved gas signal sensing module, a hydrogen (H) signal sensing module, a molecular hydrogen (H₂) signal sensing module, an ionic hydrogen (H⁺) signal sensing module, a dissolved H₂ signal sensing module, an oxygen (O) signal sensing module, a molecular oxygen (O₂) signal sensing module, a dissolved O₂ signal sensing module, a carbon dioxide (CO₂) signal sensing module, a dissolved CO₂ signal sensing module, a nitrogen (N) signal sensing module, a molecular nitrogen (N₂) signal sensing module, a hydrogen sulfide (H₂S) signal sensing module, a methane (CH₄) signal sensing module, a device signal sensing module, a computer signal sensing module, a networked computer signal sensing module, a peripheral signal sensing module, a module signal sensing module, a sensor signal sensing module, a circuit signal sensing module, a microprocessor signal sensing module, a memory signal sensing module, a communication interface signal sensing module, a bus signal sensing module, an Inter-integrated circuit (I2C) signal sensing module, a universal asynchronous receiver/transmitter (UART) signal sensing module, a serial bus signal sensing module, a universal serial bus (USB) signal sensing module, a synchronous serial connection (SPI) signal sensing module, a 1-Wire® signal sensing module, an ethernet signal sensing module, a wireless area network (WAN) signal sensing module, a WiFi signal sensing module, a Bluetooth signal sensing module, a network enabled signal sensing module, an instruction signal sensing module, an algorithm signal sensing module, a code signal sensing module, a routine signal sensing module, a subroutine signal sensing module, a firmware signal sensing module, a software signal sensing module, an error-detection signal sensing module, a checksum signal sensing module, a hash signal sensing module, a data signal sensing module, a data packet signal sensing module, an identification signal sensing module, a radio frequency identification (RFID) signal sensing module, a bar code signal sensing module and a QR code signal sensing module.

In other examples of the devices described herein, the instruction responsive module is selected from a sensor, a signal sensing module, a temperature adjustment means, a pressure adjustment means, a moving means, a pump, a syringe, a peristaltic pump, a diaphragm, a piston, a vacuum, a valve, a ball, a needle, a pressure release mechanism, a flow release mechanism, a mixer, a meter sampler, an autosampler, an alert, a light, a lamp, a light emitting diode (LED), a sound, a speaker, a horn and a buzzer.

In other examples of the devices described herein, the operation means is selected from a sensor, a moving means, a temperature adjustment means, a pressure adjustment means, an instruction responsive module, a device, a computer, a networked computer, a peripheral controlling means, a module controlling means, a circuit controlling means, an electronic controlling means, a microprocessor controlling means, a memory controlling means, a communication interface, a bus controlling means, an Inter-integrated circuit (I2C) controlling means, universal asynchronous receiver/transmitter (UART) controlling means, serial bus controlling means, a universal serial bus (USB) controlling means, a synchronous serial connection (SPI) controlling means, a 1-Wire® controlling means, an ethernet controlling means, a wireless area network (WAN) controlling means, a WiFi controlling means, a Bluetooth controlling means, a network enabled controlling means, an instruction controlling means, an algorithm controlling means, a code controlling means, a routine controlling means, a subroutine controlling means, a firmware controlling means, a software controlling means, an error-detection controlling means, a checksum controlling means, a hash controlling means, a data controlling means, a data packet controlling means, an identification controlling means, a radio frequency identification (RFID) controlling means, a bar code controlling means and a QR code controlling means.

In other examples of the devices described herein, the signal sensing module is configured to receive a signal that is generated by the sensor of the culturing device. However, the skilled person would understand that a signal sensing module is not essential, and (e.g.) a signal may be directly generated by the at least one sensor sufficient to activate the instruction responsive module directly to instruct the operation means to undertake at least one operation in accordance with the teaching provided herein.

In other examples the signal sensing module senses a signal that is selected from an electronic signal, an analog signal, a digital signal, a temperature signal, a pressure signal, a position signal, an angle or angular signal, a velocity signal, an acceleration signal, a signal from a sensor, a signal from a signal sensing module, a signal from an instruction module, a feedback signal, a data signal, a data packet signal, a module signal, an identification signal and a code signal.

In other examples of the devices described herein, the instruction generated by the instruction responsive module is selected from a digital instruction, an electronic instruction, an analog instruction, a data instruction, a data packet instruction, a module identification code instruction, a trigger instruction, an algorithm instruction, a code instruction, a software instruction, a routine instruction and a subroutine instruction.

The devices and associated analyses according to the present invention are amenable to automation and machine control. This includes automation or machine control of (i) signal inputs (i.e.) to configure or adjust or change at least one parameter of the culturing device either in response to a change in a physical or chemical property of the culture or in response to an external instruction, and (ii) signal outputs (i.e.) to record the measurement of at least one parameter associated with the culture or the culturing device, and optionally store that measurement as data in a data storage medium or feedback further signal inputs to the culturing device in response to physiological cues provided by the culture.

Accordingly, in another aspect of the present invention there is provided a method for controlling and/or adjusting at least one parameter of a culturing device comprising:
(i) inoculating a culturing device as described herein with a culture; and
(ii) activating the control module to control or adjust at least one parameter of the culturing device in response to a change in a physical or chemical property of the culture as detected by the sensor; or
(iii) activating the control module to control or adjust at least one parameter of the culturing device in response to an external instruction,
thereby controlling or adjusting at least one parameter of the culturing device.

In an example according to this and other aspects of the present invention, the step of activating the control module is achieved by way of automation or machine control.

The ability to control at least one parameter of the culturing device or to quantify or measure at least one parameter associated with the culture or culturing device is typically achieved via signal handling.

According to the present invention, signal handling comprises a signal input and/or a signal output.

In certain examples according to the present invention signal handling is selected from transmitting a signal, sending a signal, recording a signal, presenting a signal, graphing a signal, storing a signal, storing a signal on media, storing a signal in a database, storing a signal in a cloud database, storing a signal in data structures, processing a signal, signal processing, digital signal processing, encrypting a signal, analysing a signal, asynchronous phasing of a signal, synchronous phasing of a signal, sorting a signal, ranking a signal, pattern recognition of a signal, statistical management of a signal, numerical management of a signal, big data management of a signal and artificial intelligence (AI) management of a signal, which signal is an input signal to control at least one parameter of the culture or culturing device or a which signal is an output signal for the purpose of recording at least one measurement associated with the culture or culturing device.

In an example according to this and other aspects of the present invention, adjusting is selected from raising, lowering, adding, removing, extracting, changing, exchanging, agitating, mixing, consuming, culturing, feeding, incubating, growing, testing, and inhibiting at least one parameter of the culture or the culturing device.

In another example according to this and other aspects of the present invention, the signal input or signal output is an algorithm and is selected from an instruction, a set, a flowchart, a script, a software and a firmware signal.

In another aspect of the present invention there is provided a method for determining a change in a physical or chemical property of a culture comprising:
(i) inoculating a culturing device as described herein with a culture;
(ii) measuring a physical or chemical property of the culture at an initial time point;
(iii) measuring the physical or chemical property of the culture according to (ii) at a later time point;
(iv) determining any change in the physical or chemical property of the culture between the initial time point (ii) and the later time point (iii).

In another aspect, there is provided a method for reporting a signal from a culture comprising:
(i) inoculating a culturing device as described herein with a culture;
(ii) measuring a signal generated from at least one sensor of the culturing device; and
(iii) reporting the measured signal to a processing medium and/or a storage medium.

In an example according to these and other aspects of the present invention, the processing medium is selected from a computer, a computer algorithm, software, firmware, hardware, and a storage medium.

In another example according to this and other aspects the storage medium is selected from a record storage medium, a print storage medium, a paper storage medium, an electronic storage medium, an analog storage medium, a digital storage medium, a memory storage medium, a solid-state storage medium, a distributed storage medium and a cloud storage medium.

### Methods

The devices according to the present invention are particularly devised for application to gut cultures derived from any animal from the phylum Chordata, class Mammalia, suborder Ruminantia (ruminant), including domesticated and wild cattle, goat, sheep, bison, buffalo, yak, deer, antelope, and the suborder Tylopoda (pseudo-ruminants), camel, alpaca, and lamas.

In certain examples, the culture is selected from the content, extraction and excretion of a gut organ of a living or dead animal from the phylum Chordata, class Mammalia, suborder Ruminantia (ruminant) including but not limited to domesticated or wild cattle, goat, sheep, bison, buffalo, yak, deer, antelope; or the content, extraction, and excretion of a gut organ of a living or dead animal from the suborder Tylopoda (pseudo-ruminants) including but not limited to camel, alpaca and lama.

The cultures for culturing in the devices described herein may be extracted from the animal foregut, hindgut, rumen, pseudo-rumen, reticulo-rumen, omasum, abomasum, stomach, true stomach, caecum, intestine, bowel, colon, and comprise diverse material including gut content fluid, solid, semisolid, oesophageal sample, fistulated sample, rectal sample, excretion, vomit, and faeces.

The culture may also be extracted from a live animal or a deceased animal.

The culture may also be extracted from an existing culture that has been incubated or grown in a non-animal setting including but not limited to an in vitro culture, a bioreactor, an artificial rumen, and rumen simulation technique (RUSITEC).

The culture may also be derived from a storage repository including, but not limited, to a frozen culture, a chilled culture, a suspended culture and a lyophilised culture.

The culture may also be created from at least one culture comprising a pure or isolated species or strain of microorganism.

Accordingly, the present invention also contemplates methods for culturing, analysing, utilising and inhibiting the microorganism derived from the foregut organs of ruminant and pseudo-ruminant animals using the devices described herein, and includes culturing microorganism responsible for methanogenesis which represents not only lost energy but the generation of potent greenhouse gases.

The present invention further contemplates methods which simulate aspects of the gut organs of live animals, including providing in vitro models of the ruminant or pseudo-ruminant foregut, to discover, develop and assess treatments, feeds, supplements, and intra-ruminal devices or to reduce and monitor methane gas emissions from ruminant and pseudo-ruminant animals.

Accordingly, in another aspect of the present invention there is provided a method for evaluating the effect on a culture of any one or more of:
(i) a feed;
(ii) a feed supplement;
(iii) an intervention;
(iv) a medicament;
(v) microorganism;
(vi) a microorganism inhibitor;
(vii) a microorganism enhancer;
(viii) a methanogen inhibitor;
(ix) a methanogenesis inhibitor
the method comprising the steps of:
(a) inoculating a culturing device as described herein with a culture;
(b) introducing any one or more of (i) to (ix) to the culture;
(c) measuring at least one parameter associated with the culture from the at least one sensor.
thereby evaluating the effect on the culture of any one or more of (i) to (ix).

In one example according to the methods described herein, the effect of an intervention on a culture is selected from a measured change in the acidity, electrochemical potential, pH, redox potential, pressure, partial pressure, concentration, fluid, fluid displacement, gas, gas flow, dissolved gas, hydrogen (H), molecular hydrogen (H₂), ionic hydrogen (H⁺), dissolved H₂, oxygen (O), molecular oxygen (O₂), dissolved O₂, carbon dioxide (CO₂), dissolved CO₂, nitrogen (N), molecular nitrogen (N₂), ammonia (NH₃), ammonium (NH₄⁺), hydrogen sulfide (H₂S), methane (CH₄), formic acid, formate, ethanol, volatile fatty acid, short chain fatty acid, acetic acid, acetate, propionic acid, propionate, butyric acid, butyrate, isobutyric acid, isobutyrate, pentanoic acid, pentanoate, valeric acid, valerate, hexanoic acid, hexanoate, caproic acid, caproate, heptanoic acid, heptanoate, fluorescence, luminescence, co-factor F420, protein, enzyme, methyltransferase, N5-methyl-tetrahydromethanopterin: coenzyme M methyltransferase complex (mtr), methyl (alkyl)-coenzyme M reductase (MCR), gene, nucleic acid sequence, DNA sequence, RNA sequence, cDNA sequence, organism, microorganism, archaea, methanogen, bacteria and protozoa content of the culture. The effect may be an increase, decrease, stop, inhibition or no change in relation to any of the aforementioned parameters or reflect a change in an activity, function, synthesis, amount, concentration, moles, mass, number, percentage, ratio, stoichiometry and diversion of the aforementioned parameters.

In another example according to the methods described herein the intervention is selected from an adjustment, addition, administration, extraction, and subtraction, of the position, the temperature, the pressure, the pH, a feed, a supplement, a medicament, a microorganism, and inhibitor.

In another example according to the methods described herein evaluating is selected from analysed, assessed, calculated, determined, estimated, predicted, measured, modelled, simulated, validated, correlated, autocorrelated, probabilistic, statistical, numerical, graphical, algorithm, computational and artificial intelligence (AI).

In another example according the methods described herein the feed is selected from a plant, a leaf, a stem, a grain, a seed, a husk, a kernel, a pasture, a grass, a Gramineae, a legume, a Leguminosae, a clover, a lucerne, a fodder, a forage, a brassica, a herb, a chicory, a plantain, an algae, a macroalgae, a barley, a corn, a maize, a wheat, an oat, an amino acid, a peptide, a protein, a carbohydrate, a cellulose, a hemicellulose, a starch, a fat and an oil.

In another example according to the methods described herein the feed is selected from a cut feed, a chopped feed, a ground feed, a preserved feed, a straw feed, a hay feed, a silage, baleage, a frozen feed, a chilled feed, a dried feed, a freeze dried feed, a rolled feed, a crushed feed, a ground feed, a pelleted feed, a tableted feed and a cooked feed.

In another example according to the methods described herein the feed is selected from a digested feed, a pre-digested feed, a treated feed, a pre-treated feed, an enzyme-treated feed, an acid-treated feed and an incubated feed.

In another example according to methods described herein said feed supplement is selected from a vitamin, a drug, a chemical, an element, a trace element, a probiotic, a prebiotic, a postbiotic, a plant, an extract, an algae, a mineral, a trace element, a top dressing, a fluid, a solid, a lozenge, a capsule and a bolus.

In another example according to the methods described herein the medicament is selected from an agent, an adjuvant, a drug, a chemical, a biochemical, a biomolecule, a nucleic acid, an amino acid, a peptide, a protein, a pharmaceutical, a nutraceutical, a therapeutic, an antibiotic, an anthelminthic, an ionophore, an antiprotozoal, a defaunation agent, a fungicide, a fluid, a solid, a lozenge, a bolus, an electronic bolus and a device.

In another example according to the methods described herein the microorganism is selected from a probiotic, a bacterium, an archaea, a fungi, a protozoa, a parasite, a helminth, a nematode, a cestode, a trematode, a virus, a phage, a spore, an egg and an oocyte.

In another example according to the methods described herein said microorganism inhibitor is selected from a drug, a chemical, an agent, an antibiotic, an antiprotozoal, a defaunation, an ionophore, an anthelminthic, an antifungal, a fungicide, a fluid, a solid, a lozenge, a bolus, an electronic bolus and a device.

In another example according to the methods described herein said microorganism enhancer is selected from a carbohydrate, a sugar, glucose, glycerol, an oil, a vegetable oil, canola oil, olive oil, mineral oil, paraffin oil, a fat, a lipid, a liposome and a triacyl glyceride.

In another example according to the methods described herein the methanogen inhibitor is selected from a chemical, a compound, a drug, a gas, a liquid, an encapsulation, a liposome, a lipid nanoparticle, oxygen (O₂), ozone (O₃), peroxide, calcium peroxide, an antibiotic, an organohalogen, a haloform, chloroform, bromoform, iodoform, a seaweed, Asparagopsis sp., Asparagopsis extract, Asparagopsis matter, an alliin, an iso-alliin, an allicin, Mootral™, garlic extract, garlic matter, 3-Nitrooxypropanol (3-NOP), Bovaer™, a fluid, a solid, a lozenge, a bolus, a device, an electronic bolus, light, biocidal light, flavin, Co-factor F420, photobleaching agent, 420 nm light.

In another example according to the methods described herein the methanogenesis inhibitor is selected from a chemical, a compound, a drug, a gas, a liquid, an encapsulation, a liposome, a lipid nanoparticle, oxygen (O₂), ozone (O₃), peroxide, calcium peroxide, an antibiotic, an organohalogen, a haloform, chloroform, bromoform, iodoform, a seaweed, Asparagopsis sp., Asparagopsis extract, Asparagopsis matter, an alliin, an iso-alliin, an allicin, Mootral™, garlic extract, garlic matter, 3-Nitrooxypropanol (3-NOP), Bovaer™, a fluid, a solid, a lozenge, a bolus, a device, an electronic bolus, light, biocidal light, a Co-factor F420, photobleaching agent, 420 nm light.

Despite being well adapted to the culture of microorganisms derived from the foregut and gut of ruminant and pseudo-ruminant animals, the present invention may also be applied to a gut culture derived from a non-ruminant animal, including, but not limited to, a gut culture from an avian (bird), a canine (dog), an equine (horse), a feline (cat), a porcine (pig) and a primate, (e.g. human, ape, monkey).

### Further Background on Gut Fermentation Biology

Microorganisms in the gut of animals derive energy and metabolites from materials consumed by the animal (as feed), materials produced by the animal (e.g. mucus and sloughed cells from the gastrointestinal tract), and microorganism produced materials (e.g. microbial metabolites, including microorganism predation). Some microorganisms produce enzymes not produced by animals, which facilitate the disassembly of long chain carbohydrates including for example cellulose, hemi-cellulose, and starch, which are broken into five and six carbon sugars and then metabolized to molecules including VFAs, CO₂, and metabolic hydrogen. The action of microorganisms on carbohydrates predominantly in the absence of O₂ is called fermentation, which also produces heat, and in the gut content of an animal is called enteric fermentation. The VFAs produced by enteric fermentation comprise carboxylic acid compounds from 1 to 7 carbons in straight and branched isomeric forms and include formic, acetic, propionic, butyric, isobutyric, valeric, isovaleric, 2-methybutyric, hexanoic, and heptanoic acids.

For example, the VFAs acetate, propionate, and butyrate are produced from the six-carbon sugar glucose according to the chemical reactions:

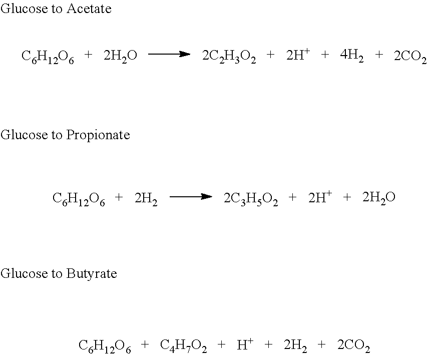

The VFAs are weak acids with pka of ≤4.8 and so are predominantly (90 to 99%) present as their anionic forms in the healthy gut at near neutral pH and include acetate, propionate, butyrate, isobutyrate, valerate, isovalerate, 2-methybutyrate, hexanoate, and heptanoate. The VFAs produced by enteric fermentation can be utilised by the animal as an energy source via absorption from the gut through the gut epithelial cells. Such absorption provides a thermodynamic sink for the VFAs and without such a VFA sink the chemical reactions that yield the VFAs would approach chemical equilibrium and the increasing concentration of VFAs in the gut content would cause the inhibition of their synthesis. The VFA sink in the gut is augmented by anatomical features that increase the surface area of the gut and the absorption capacity of the gut (i.e. ruminal papillae and epithelial cilia). In certain examples according to the present invention, the culturing devices are configured to mimic these physiological processes by providing a VFA adsorption means, optionally within the conduit, to simulate the adsorption of VFAs by ruminal papillae and epithelial cilia).

Thus, the culture of microorganisms in the animal gut provides the animal the advantage of utilising otherwise indigestible feed materials for maintenance and growth.

In the microorganism production of VFAs, metabolically active hydrogen atoms are bound to hydride shuttle molecules (e.g. flavin and nicotinamide co-factors) and then removed by hydrogenase enzymes to regenerate the shuttle and molecular hydrogen (H₂) is produced. The H₂ is dissolved H2 (dH₂) in the gut liquor, which can nucleate and forms bubbles containing gaseous H2 (gH₂). Accordingly, in other examples according to the present invention, the culturing devices are configured to include a dissolved hydrogen sensor which measures the amount of dissolved hydrogen in the liquor to reflect these metabolic processes.

CO₂ is produced by the incomplete oxidation of carbohydrates via enzymes that facilitate decarboxylation reactions to yield CO₂ as dissolved CO₂ (dCO₂) in the gut liquor, which can nucleate and form bubbles containing gaseous CO₂ (gCO₂). In some animal gut organs, methanogenesis is facilitated by hydrogenotrophic methanogenic archaea, which consume dH₂ in the reduction of CO₂ to CH₄ according to the general reaction:

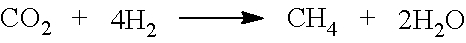

The CH₄ is produced as dissolved CH₄ (dCH₄) in the gut liquor which can nucleate and form bubbles containing gaseous CH₄ (gCH₄).

Inhibition of methanogenesis causes changes to metabolic products of enteric fermentation including changes to the amount and distribution of VFAs, a decrease in CH₄ and an increase in CO₂ and H₂.

Inhibition of methanogenesis can occur by treatment of a microorganism culture with a dosage of a material capable of inhibiting an enzymic process required for methanogenesis or which can inhibit and/or kill a methanogenic organism.

All mammalian animals appear to exhibit some capacity for enteric fermentation of materials by microorganisms in various organs of the gut for the production of VFAs, and potentially producing CO₂, H₂ and CH₄.

Fermentation that occurs in gut organs anterior to the acid-secreting stomach organ of the gastrointestinal tract is called foregut (forestomach) fermentation, whereas hindgut fermentation occurs in organs post the stomach.

While most mammals exhibit hindgut fermentation, some types of herbivorous animals have evolved specialised foregut organs that extract energy from the fermentation of consumed plant material and so exhibit both foregut and hindgut fermentation.

In ruminant animals the sites of fermentation are foregut organs including the rumen and reticulum, which are anterior to the acid-secreting abomasum (also called the true stomach). The foregut organs, predominantly the rumen and reticulum (also called the reticulorumen, herein called the rumen) are responsible for approximately 90-95% of the enteric fermentation and the resulting gases can be emitted from the animal by a process of eructation.

The hindgut organs are posterior to the abomasum and include the caecum and large colon. Hindgut fermentation gases are emitted from the animal by a process of flatulence.

The acidity of the abomasum due to the secretion of hydrochloric acid (HCl) is lowered to pH ~2 is hostile to rumen microorganisms and together with secreted enzymes causes the killing, digestion and subsequent absorption of microbial biomass by the animal. Consequently, the culture in the foregut is different to the culture in the hindgut.

Ruminant animals are animals from the suborder Ruminantia and include for example cattle, goat, sheep, bison, buffalo, yak, deer, and antelope.

Other animals that exhibit foregut fermentation are the pseudo-ruminants, which are animals from the suborder Tylopoda and include for example camel, alpaca, and lama.

The hippopotamus, some species of monkey, and marsupials also exhibit foregut fermentation.

Herbivorous animals that only exhibit hindgut fermentation include for example rodents, lagomorphs, horses, and elephants.

Omnivorous animals are known to exhibit hindgut fermentation and include for example pigs, apes, and humans.

Carnivorous animals are known to exhibit hindgut fermentation and include for example cats, and dogs.

Avian, amphibian, and reptilian animals are known to exhibit hindgut fermentation and include for example birds, frogs, toads, lizards.

The culturing devices and methods described herein are intended to provide a model system which more accurately mimics animal gut organ physiology to discover, develop, and assess feeds, treatments, additives, supplements, and devices which ultimately improve the health and productivity of animals in farming practices and help reduce greenhouse gas emissions which contribute to climate change issues.

The invention is further described with reference to the following examples. It will be appreciated that the invention as claimed is not intended to be limited in any way by these examples.

### EXAMPLES

### Example 1: Exemplary Culturing Devices

The present invention was conceived to meet the need for a low cost, simple to operate animal gut fermentation system that is without the disadvantages and limitations of prior art approaches, which disadvantages and limitations include:
(i) mixing mechanisms that intentionally or inadvertently disrupt the stratification of the liquor, including the interface between the liquor and the headspace and the structure of the fibre mat, which has the disadvantage that the integrity of the culture is compromised and contributes errors and uncertainty in the measurements of enteric fermentation;
(ii) flushing the headspace with CO₂ and/or N₂ to promote anaerobic conditions widely believed to be essential for methanogenesis, which flushing incurs the requirement for gas procurement and additional gas delivery infrastructure and has the disadvantages of requiring specialist expertise to operate and undesirable additional costs; and
(iii) vacuum pumping the liquor to remove sedimentary or waste materials, which pumping incurs the requirement for additional liquid handling infrastructure and has the disadvantages of being prone to blockage and requiring specialist expertise to operate and undesirable additional costs.

The present invention reports the finding that microorganisms of gut organs of animals can be cultured in a device that unlike other devices, retains the integrity of the interface between the liquor and the headspace and the structure of the fibre mat, does not require the additional complexity and costs of gas flushing the headspace with CO₂ and/or N₂, or the problems and costs of vacuum pumping of the liquor. The culturing devices according to the present invention are amenable to automation and comparatively simple to operate and displays methanogenesis profiles remarkably like the those of live animals.

The present invention comprises a vessel to contain a culture, which vessel is fluidically connected to at least one conduit. The vessel may contain a removeable VFA sink optionally located in the conduit, is pressure controlled and may be moved by a control module about an axis of rotation to prescribed positions, angles, and orientations with prescribed accelerations, velocities, with prescribed intervals of movement interspersed with prescribed intervals of stillness causing the culture inside the vessel to be subjected to prescribed temperatures, prescribed pressures, and prescribed motion, agitation and mixing, to controllably contact, enter, and exit the conduit according to prescribed movement to change at least one parameter of the culture and to permit analysis of the fermentation profile.

The vessel may optionally contain a temperature adjustment means, although the skilled person would recognise that this feature is optional for the reason that a culturing device according to the present invention may be housed in a temperature-controlled room or space.

FIG. 1 depicts an example of a culturing device 100 shown in schematic transverse cross-section. The culturing device 100 includes a vessel 101 with vessel walls 102 for containing a culture 103 comprising a liquor 104 and a headspace 105 separated by an interface 106. The culturing device 100 includes at least one sensor 107 in contact with the culture 103 being in contact with the liquor 104 and/or the headspace 105 and/or the interface 106. The temperature of the culture 103 is adjusted by temperature adjustment means 108 applied directly to at least one vessel wall 102, although the temperature adjustment feature is considered optional. Connected to the vessel 101 is at least one conduit 109a and/or 109b, which has at least one external opening 110ah and/or 110av and/or 110b, at least one internal opening 111a and/or 111b, a conduit wall 112a and/or 112b, and a conduit lumen 113a and/or 113b fluidically connected to said vessel 101 providing a path 114a and/or 142b for the culture 103 being predominantly a path 114a for the liquor 104 and/or a path 114a for the headspace 105. The culturing device 100 includes a pressure adjustment means 118 for adjusting the pressure of the culture 103, moving means 120 for moving the vessel 101 about an axis of rotation 121 and thereby moving the culture 103 and a VFA adsorption means 122 for reducing the concentration of VFAs in the culture 103.

The culturing device 100 includes a control module 125.

In some examples the control module 125 is provided to control at least one component of the culturing device 100 selected from the group comprising a sensor 107, a temperature adjustment means 108, a pressure adjustment means 118, and a moving means 120.

In some examples the vessel walls 102 surround and contain the culture 103 comprising the liquor 104 being predominantly the volume of liquid fluids containing solids, semisolids, gas bubbles, and dissolved gases, and the headspace 105 being that volume above the liquor 104 and predominantly containing gas and vapor fluids.

In some examples at least one vessel wall 102 can be detached to open the vessel 101 and replaced to close the vessel 101.

In some examples at least one vessel wall 102 is selected from the group comprising rigid, flexible, elastic, conductive, insulating, optically opaque, optically transparent, biocompatible.

In some examples the materials of at least one vessel wall 102 are selected from the group comprising polymer, polymethyl methacrylate (PMMA, acrylic, Perspex), polycarbonate, polyvinyl chloride (PVC), polystyrene, acrylonitrile butadiene styrene (ABS), metal, steel, stainless steel.

In some examples the inside surface of the vessel walls 102 contacting the culture 103 can be textured to modify the adsorption of culture 103 by a method selected from the group comprising roughening, smoothing, polishing, coating, chemical treatment.

In some examples the vessel 101 is a box geometry as depicted 100. In some examples the vessel 101 is any geometry to efficiently contain the volume of the culture 103.

In some examples the vessel 101 is fluid tight to prevent the leakage of fluids including liquids and gases.

It will be appreciated that the movement of the vessel 101 will cause the movement of the culture 103 and that the location of the axis of rotation 121 combined with the shape of the vessel 103, the intervals of movement, the intervals of stillness, the speed, the acceleration, and the angles of rotation will permit the movement and the mixing of the culture 103 to be prescribed, adjusted, and optimised to model the movements and mixing of the gut content of the live animal to preserve the structure and integrity of the culture 103, the liquor 104, the headspace 105 and the interface 106.

In some examples the vessel 101 includes at least one fixed and/or unfixed geometric shape feature to modulate the movement of the culture 103 within the vessel 101 selected from the group consisting of insert, baffle, and curvature.

In some examples at least one vessel wall 102 can be insulated to limit the flux of heat in and out of the vessel 101 to prevent the temperature of the culture 103 from undesirable fluctuation.

The culturing device 100 is scalable at different size scales. In some examples the culturing device 100 can be constructed with a vessel 101 scaled to model a specific animal gut organ. For example, a sheep has a rumen volume of about 0.5 L to about 6 L, and a cattle has a rumen volume of about 5 L to about 100 L. The high variability of foregut organ size depends on the species, breed, sex, and age of the animal. In some examples the vessel 101 has a total internal volume selected from the group comprising about 50 mL, about 500 mL, about 1 L, about 2 L, about 5 L, about 10 L, about 50 L, about 75 L, about 100 L, and about 150 L. It will be appreciated that by scaling the size of the vessel 101 naturally requires comparable scaling of temperature adjustment means 108, the pressure adjustment means 118, the conduit 109a and/or 109b. the VFA adsorption means 122 and the movement means 120.

The culturing device 100 includes at least one sensor 107.

In some examples the sensor 107 is attached to or inside the vessel 101 and passing through at least one vessel wall 102 as depicted 100. Alternatively, at least one sensor may be positioned in any part of the culturing device selected from the group consisting of the vessel 101, vessel wall 102, conduit 109a and/or 109b, conduit wall 112a and/or 112b, conduit lumen 113a and/or 113b, temperature adjustment means 108, pressure adjustment means 118, and moving means 120.

In some examples the sensor 107 detects properties of the vessel 101 selected from the group consisting of position, velocity, acceleration, pressure, temperature, chemical composition, concentration, mass, and volume.

In some examples the sensor 107 detects the gas products in or produced from the culture 103. In some examples the gases are emitted into the headspace 105 from the liquor 104 and the sensor 107 is located in the headspace 105 or a conduit 109a and/or 109b connected to the headspace 109b or the conduit headspace 116 to detect a gas selected from the group comprising CH₄, CO₂, H₂, H₂S, N₂, NH₃, O₂. In some examples the gases are gases dissolved in the vessel liquor 104 and the sensor 107 is a dissolved gas sensor located in the liquor 104 or the conduit liquor 115 and configured to detect a dissolved gas selected from the group comprising CH₄, CO₂, H₂, H₂S, N₂, NH₃, O₂. In some examples a sensor 107 may detect a gas in the headspace 105 and a dissolved gas in the liquor 104 to determine the ratio of gas to dissolved gas inside the vessel 101.

In some examples the sensor 107 detects physical properties of the headspace 103, or conduit headspace 116 or the liquor 103 or the conduit liquor 115 from the group consisting of temperature, pressure, volume, mass, density, and specific gravity.

In some examples the sensor 107 detects chemical properties of the headspace 103, or the conduit headspace 116 or the liquor 103 or the conduit liquor 115 selected from the group consisting of acidity, pH, electrochemical potential, reduction oxidation potential, and Eh.

In some examples the sensor detects optical properties of the headspace 103, or the conduit headspace 116 or the liquor 103 or the conduit liquor 115 from the group comprising absorbance, bioluminescence and fluorescence.

The culturing device 100 includes temperature adjustment means 108.

In some examples the temperature adjustment means 108 comprises a controllable temperature adjustment apparatus attached to at least one vessel wall 102 and/or inserted inside the vessel 101 to heat and/or cool the culture 103, which may be necessary to efficiently culture microorganisms at a predetermined temperature.

In some examples the culture 103 can be maintained at a desired temperature by temperature adjustment means 108 selected from the group consisting of temperature sensor, heater, cooler, thermistor, thermocouple, heater element, Peltier device, water jacket, and fan.

At least one sensor 107 can be configured to contact the culture 103 so that the temperature adjustment means 108 working in concert with the sensor 107 can maintain the culture 103 at a predetermined temperature.

In some examples the culture 103 temperature is predetermined by the temperature most efficient for culturing microorganisms from the foregut of ruminant animals, which is typically about 39° C. In some examples there may be need to adjust the culture 103 to a temperature selected from the group consisting of about freezing, about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., and about 50° C.

It will be appreciated that the fermentation of feed by the culture 103 will produce heat that may change the temperature of the culture 103. Maintaining the temperature of the culture 103 by the temperature adjustment means 108 within predetermined limits is required to faithfully culture the microorganisms from an animal gut, particularly the ruminant animal foregut.

In some examples the heat produced by the culture 103 will be determined by comparing the thermal characteristics of the vessel 101 maintained at least one temperature with and without a culture 103.

The culturing device 100 includes pressure adjustment means 118.

In some examples the pressure adjustment means 118 comprises a controllable pressure adjustment apparatus attached to at least one conduit 109b as depicted 100, or alternatively to at least one conduit 109a and/or 109b, attached to or through a vessel wall 102 and/or inserted inside the vessel 101 to raise and/or lower the pressure of the culture 103, which may be necessary to efficiently culture microorganisms at a predetermined pressure.

In some examples the culture 103 can be maintained at a desired pressure by pressure adjustment means 118 selected from the group consisting of an airlock, a pressure sensor, valve, regulator, pump, vacuum, compressor, compressed, air, and gas.

At least one sensor 107 can be configured to contact the culture 103 so that the pressure adjustment means 118 working in concert with the sensor 107 can maintain the culture 103 at a predetermined pressure.

In some examples the culture 103 pressure is predetermined as that pressure most efficient for culturing microorganisms from the foregut of ruminant animals, which is typically about near atmospheric pressure. In some examples there may be need to adjust the culture 103 to a pressure selected from the group consisting of atmospheric pressure, near atmospheric pressure, less than atmospheric pressure, and greater than atmospheric pressure.

It will be appreciated that the fermentation of feed by the culture 103 will produce gases that are emitted from the liquor 104 into the headspace 105 and may change the pressure of the culture 103 including the pressure of the headspace 105. Maintaining the pressure of the culture 103, particularly the pressure of the headspace 105 by the pressure adjustment means 118 within predetermined limits is required to faithfully culture the microorganisms from an animal gut, particularly the ruminant animal foregut.

In some examples, the culturing device 100 includes VFA adsorption means 122.

In some examples the VFA adsorption means 122 is located in the conduit 109a and contacting the conduit liquor 115 as depicted in FIG. 1 100, In some examples the VFA adsorption means 122 is located in the vessel 101 and contacting the liquor 104. In some examples thew VFA adsorption means is partly located in the conduit 109a and partly located in the vessel 101 and partly contacting the conduit liquor 115 and the liquor 104. The use the conduit 109a to locate the VFA adsorption means 122 in either the conduit 109a or in the vessel 101 has the advantage that the VFA adjustment means 122 can be conveniently inserted, extracted, and replaced.

In some examples the VFA adjustment means 122 comprises components selected from the group comprising resin, ion exchange resin, anion exchange resin, weak anion exchange resin, strong anion exchange resin cation exchange resin, weak cation exchange resin, strong cation exchange resin, filter, housing, and membrane.

In some examples the VFA adjustment means 122 adjusts the concentration of VFA in the culture 103 to reduce the concentration of at least one VFA selected from the group comprising acetate, propionate, butyrate, isobutyrate, pentanoate, valerate, isovalerate, hexanoate, caproate, and heptanoate.

It will be appreciated that the fermentation of feed by the culture 103 may produce VFAs that are dissolved the liquor 104 and may change the VFA concentration of the culture 103 including the VFA concentration in the liquor 104. Reducing the VFA concentration of the culture 103, by the VFA adsorption means 122 is required to provide a VFA sink to faithfully culture the microorganisms from an animal gut, particularly the ruminant animal foregut.

Inside the vessel 101, the liquor 104 and the headspace 105 meet at a liquid-gas interface, herein call the interface 106. The interface 106 is depicted as a wavy line in two dimensions but in three dimensions is a roughly planar interface 106 and is about co-planar with the earth's mean horizon and called level. It will be appreciated that the interface 106 will equilibrate to level irrespective of the orientation of the vessel 101 such that when the vessel 101 is moved the interface 106 will rapidly adjust to maintain its level with the earth's mean horizon.

The volume of the liquor 104 and the volume of the headspace 105 determines the level height of the interface 106. In some examples the volume of the liquor 104 as a percentage of the total volume of the vessel 101 is selected from the group comprising: about 20 percent, about 50 percent, about 75 percent, and about 90 percent.

The composition of the liquor 104, the composition of the headspace 105 and the composition of the interface 106 are co-dependent and dominated by physical and chemical properties of the culture 103 including temperature, pressure, and viscosity of the fluids, the chemical and particulate composition of the liquor 104, the microorganism diversity of the culture 103, the concentrations of dissolved gases in the liquor 104, the partial pressures of individual gases in the headspace 105, the material structure and mechanical complexity of the interface 106, and the surface tension of the interface 106. Thermodynamic principles will dictate whether a gas in the liquor 104 will potentially contact the interface 106 and cross (emit) from the liquor 104 into the headspace 105 or whether a gas in the headspace 105 will potentially contact the interface 106 and cross from the headspace 105 and cross (dissolve) into the liquor 104. The interface 106 is an important structural feature of the gut culture in a ruminant animal foregut and in a rumen is called the mat, or fiber mat, on account of its dense fibrous composition and complexity, which separates the rumen liquor from the rumen headspace, otherwise called the gas cap, on account that it accumulates fermentation gases. It will be appreciated that preserving the composition, mechanical properties, and thermodynamic characteristics of a culture of a ruminant animal foregut is an important function of the present invention of a culturing device 100 for culturing microorganisms from an animal gut.

The culturing device 100 includes at least one conduit 109a and/or 109b that provides a path for the culture 103.

In some examples the vessel 101 includes at least one conduit 109a that provides a path for the culture 103 predominantly the liquor 104.

The conduit 109a has depicted two external openings 110av and 110ah which provide two alternative paths to the conduit lumen 113a and ultimately the culture 103 predominantly the liquor 104. The external opening 110av being a vertical opening that is coaxial with the conduit 109a to assist with the administration and withdrawal or extraction of materials and components or component parts into the conduit 109a, and an external opening 110ah that is not coaxial to the conduit 109a and for example as depicted in FIG. 1 100 as normal to the conduit 109a to assist in the extraction materials from the conduit 109a.

In some examples the vessel 101 includes at least one conduit 109b that provides a path for the culture 103 predominantly the headspace 105. The conduit 109b is depicted as a path for the headspace 105 which can direct the gases to and from the headspace 105 via the conduit 109b for gas addition, removal, analysis, treatment, capture, and disposal.

In some examples at least one conduit 109a and/or 109b is connected to the vessel 101 via entry through the vessel wall 102.

In some examples the conduit 109a is a tube comprising a conduit wall 112a encasing a conduit lumen 113a that fluidically contacts the liquor 104 below the level height of the interface 106 providing a path 114a to and/or for the liquor 104.

In some examples the conduit 109a can provide a path 114a to and/or for the liquor 104 via the internal conduit opening 111a and/or the conduit 109b can provide a path 114b to and/or for the headspace 105 via the internal conduit opening 111b.

In some examples the conduit 109b is a tube comprising a conduit wall 112b encasing a conduit lumen 113b that fluidically contacts the headspace 105 above the level height of the interface 106 providing a path 114b to and/or for the headspace 105 via the internal conduit opening 111b.

The fluidic contact of the conduit 109a with the culture 103 causes the conduit lumen 113a to contain conduit liquor 115, a conduit headspace 116, and a conduit interface 117. The physics of the system causes the conduit interface 117 to equilibrate via the path 114a with the interface 106 so that are both the conduit interface 117 and the interface 106 level and co-planar.

It will be appreciated that the liquor 104 in the vessel 104 is in fluidic communication with the conduit liquor 115 in the lumen 113a of the conduit 109a.

In some examples the conduit wall 112a and/or 112b can be insulated to limit the flux of heat in and out of the conduit 109a and 109b to prevent the temperature of the culture 103 from rapid or undesirable fluctuation.

In some examples the conduit wall 112a and/or 112b can include temperature adjustment means.

In some examples the diameter and length of the conduit 109a and 109b is selected so that the percentage of culture 103 that that occupies the conduit lumen 113a and/or 113b at any one time is selected from the group comprising about 0%, about 0.1% about 0.5% about 1%, about 5%, about 10%, about 15%, about 20%, about 25% about 30%, about 40%, about 50%, about 60%, about 70%, about 80%, and about 90%.

In some examples the conduit external opening 110ah and/or 110av and/or 110b provides access to the conduit lumen 113a and/or 113b, the conduit headspace 116, the conduit interface 117, and the conduit liquor 115 and by hydraulic contact the culture 103, the liquor 104, the interface 106, and the headspace 106.

It will be appreciated that the conduit 109a and/or 109b provides a route for materials into and out of the culture 103 whether it be a conduit 109a to the liquor 104 or a conduit 109b to the headspace 105.

In some examples the conduit 109a and/or 109b can be adjusted from fully open to fully closed including partially open or partially closed.

In some examples the external conduit opening 110ah and/or 110av and/or 110b can be adjusted from fully open to fully closed including partially open or partially closed.

In some examples the internal conduit opening 111a and or 111b can be adjusted from fully open to fully closed including partially open or partially closed.

In some examples the conduit lumen 113a and/or 113b can be adjusted from fully open to fully closed including partially open or partially closed.

It will be appreciated that by adjusting the conduit 109a and/or 109b from fully open to fully closed including partially open or partially closed limits or permits materials entering and/or leaving the vessel 101.

In some examples the conduit lumen 113a and/or 113b can contain a permanent or temporary component to control the path 114a and/or 114b such as static component such as an insert or dynamic component such as a tap or a valve that can occlude or partially occlude the path 114a and/or 114b. In some examples the component can include an auger or feed screw that can be manual or motorised which can be operated unidirectionally or bi-directionally to control the path 114a and/or 114b.

In some examples a separate tube with a diameter smaller than the diameter of the conduit 109a and/or 109b can be inserted into the conduit lumen 113a and/or 113b and located at a predetermined location within the conduit 109a and/or 109b or the vessel 101 wherein the separate tube can be used to extract and/or administer materials.

In some examples the conduit 109a and/or 109b holds a component or component part to measure a parameter of the culture 103 such as a parameter of the liquor 104 or a parameter of the headspace 105 selected from the group consisting of temperature, acidity, pH, electrochemical potential, reduction oxidation potential, and Eh. Such components or component parts held in the conduit 109a and/or 109b are selected from the group consisting of sensor, temperature sensor, temperature probe, thermocouple, thermistor, electrochemical sensor, pH probe, Eh sensor, Eh probe.

In some examples the conduit 109a and/or 109b holds a component or component part to extract a material from the culture 103 such as a material from the liquor 104 selected from the group consisting of ion, anion, carboxylic acid anion, volatile fatty acid anion, acetate, propionate, butyrate, isobutyrate, pentanoate, valerate, hexanoate, caproate, heptanoic acid, heptanoate, cation, and NH₄⁺. Such components or component parts are selected from the group consisting of resin, ion exchange resin, anion exchange resin, weak anion exchange resin, strong anion exchange resin cation exchange resin, weak cation exchange resin, and strong cation exchange resin.

In some examples the conduit 109a and/or 109b holds a component or component part to extract a material from the culture 103 such as a material from the headspace 105 selected from the group consisting of gas, vapor, particle, H₂S, H₂O, and dust. Such components or component parts are selected from the group consisting of filter, tube, scrubber, gas scrubber, H₂S scrubber, H₂O trap, iron, steel, and steel wool.

The culturing device 100 includes moving means 120 to move the vessel 101.

In some examples a cradle is provided for holding the vessel 101 which cradle is fixed to a chassis and the moving means 120 is applied to the cradle.

In some examples the moving means 120 can move the vessel 101 about at least one axis of rotation 121.

In some examples the axis of rotation 121 can be fixed by contact through an axle to provide symmetrical rotation alternatively or the axis of rotation 121 can move such as by contact through a cam or a cam gear to provide asymmetric rotation.

In some examples the moving means 120 includes an actuator that performs a mechanical displacement, which moves the vessel 102 about the axis of rotation 121. In some examples the moving means 120 is selected from the group comprising manual movement, mechanical movement, movement about a pivot, axle movement, uniaxial movement, biaxial movement, triaxial movement, motor-driven movement, electric movement, alternating current (AC) movement, direct current (DC) movement, stepper movement, servo movement, direct-drive movement, geared movement, belt drive movement.

In some examples the axis of rotation 121 may be inside the profile of the vessel 121 as depicted 100 or alternatively the axis of rotation 121 may be outside the profile of the vessel 121 including above, below, or beside the profile of the vessel 121.

In some examples the axis of rotation 121 is configured to be at the centre of gravity of the vessel 101 to reduce the work required by the moving means 120 and reduce wear and tear on the moving means 120. In some examples the centre of gravity of the vessel 101 can be adjusted by adding or removing counterweights to the vessel as may be required if for example the mass of the vessel 101 is adjusted.

In some examples the moving means 120 may include friction reduction to reduce wear and tear on the moving means 120 selected from lubricant, bearing, sealed bearing.

In some examples, the moving means can specify intervals of movement and intervals of stillness, and to specify the speed, acceleration, and angles of rotation.

The moving means 120 moves the vessel 101 from a first position for an interval of time and to at least one second position for an interval of time.

In some examples the vessel 101 is moved clockwise or counterclockwise about an axis of rotation 121 to at least one first predetermined position and after a predetermined interval of time moved clockwise or counterclockwise about an axis of rotation 121 to at least one second predetermined position.

In some examples a process of movement may be repeated for a set interval of time or for a set number of repetitions.

The present invention provides a vessel 101 caused to move by the moving means 120 about an axis of rotation 121, which in turn causes moving of at least one portion of the culture 103 between the vessel 101 and the conduit 109a and/or 109b to change at least one parameter of the culture 103.

In some examples moving is selected from a clockwise or counterclockwise adjustment about the axis of rotation 121 from at least one first position to at least one second position of about 1 degree, about 5 degrees, about 10 degrees, about 15 degrees, about 20 degrees, about 30 degrees, about 40 degrees, about 50 degrees, about 60 degrees, about 70 degrees, about 80 degrees, about 90 degrees, about 100 degrees, about 110 degrees, about 120 degrees, about 130 degrees, about 140 degrees, about 150 degrees, about 160 degrees, about 170 degrees and about 180 degrees.

In some examples the moving means 120 can be prescribed by a parameter selected from the position, angle, direction, velocity, angular velocity acceleration, angular acceleration.

In some examples the intervals of stillness between movement can be prescribed wherein the vessel 101 is stationary for intervals of time selected from about 1 second, about 10 seconds, about 30 seconds about 1 minute, about 2 minutes, about 5 minutes about 10 minutes about 30 minutes, about 1 hour, about 2 hours, about 6 hours.

In some examples the movement and intervals of stillness can be the same of different.

In some examples the movement of the vessel 101 about the axis of rotation 121, the adjustment of position including scale and direction, the parameters of moving means 120, and the prescribed intervals of stillness can be performed by machines with precision and repeatability and is amenable to automation.

It will be appreciated that moving the vessel 101 about the axis of rotation 121 will also cause the culture 103 including the liquor 104 to move inside the vessel 101 and the conduit liquor 115 to move inside the conduit lumen 113a and the fluidic contact through the internal conduit opening 111a and/or 111b causes the flow and transfer of culture between the vessel 101 and the conduit 109a and/or 109b and vice versa depending on the movement. The movement of the vessel 101 causes mixing of the culture 103 between the conduit 109a and/or 109b and the vessel 101 and that by controlling the movement and intervals of stillness permits the control of mixing between the conduit 109a and/or 109b and vessel 101 and preserve the structure of the fibre mat at the interface 106.

In some examples the external conduit opening 110ah and/or 110av and/or 110b can be open, partially open, or fully closed, which when the vessel is caused to move may limit the hydraulic flow and transfer of culture 103 between the vessel 101 and the conduit 109a and/or 109b. It will be appreciated that adjusting the external conduit opening 110ah and/or 110av and/or 110b permits an additional level of adjustment of the mixing between the conduit 109a and/or 109b and vessel 101.

In some examples materials administered to the conduit 109a and/or 109b can be controllably introduced into the vessel 102 by specifying the parameters of movement and intervals of stillness with precision and repeatability and is amenable to automation. Materials may be transferred from the group comprising fluids, gases, dissolved gases, liquids, water, buffer, solid, feeds, feed supplements, devices, probes sensors.

It will be appreciated that by moving the vessel 101 will cause the conduit liquor 115 and the conduit interface 117 to move and that beyond a critical angle the conduit liquor 115 will be caused to pour from the external conduit opening 110ah and/or 110av and/or 110b and that by controlling the movement and intervals of stillness provides for precise and repeatable pouring of liquor 103 from the vessel 101, which is amenable to automation.

In some preferred examples moving the vessel 101 will cause the conduit liquor 115 and the conduit interface 117 to move to a critical angle that whereby the conduit liquor 115 will be caused to pour only from the external conduit opening 110ah.

It will be further appreciated that by pouring the conduit liquor 115 by controlling the movement and intervals of stillness provides for preferential decanting of the vessel liquor 104 whilst retaining within the vessel 102 selected components of the vessel liquor 104.

In some examples the parameter of the culture 103 that can be changed due to the movement of the culture between the vessel 101 and the conduit 109a and/or 109b can include the position of the culture 103 including level, distribution, volume of the liquor 104, the headspace 105, or the interface 106, the composition of the culture 103 including the composition of the liquor 104, the headspace 105, or the interface 106, or the temperature of the culture 103 including the temperature of the liquor 104, the headspace 105, or the interface 106.

In some examples the control module 125 is provided to record at least one signal from at least one component of the culturing device 100 selected from the group comprising sensor 107, temperature adjustment means 108, pressure adjustment means 118, moving means 120.

In some examples the control module 125 is provided to record at least one signal from the culturing device 100 selected from the group comprising the setting of temperature adjustment means 108, the actual temperature, the setting of pressure adjustment means, the actual pressure, the setting of movement means, parameters of movement, intervals of stillness, the actual position, the status of at least one sensor 107, and data from at least one sensor 107.

In some examples the control module 125 controls at least one culturing device 100.

In some examples the control module 125 controls the culturing device 100 by communications selected from the group comprising hard wired, optical fibre, wireless, via a network, via a telecommunications network.

In some examples the control module 125 controls more than one culturing device 100 in one or more geographic locations.

In some examples the control module 125 comprises at least one control module 125 or may comprise many levels of control module 125 forming a hierarchy of control modules.

In some examples the control module 125 is selected from the group comprising controlled, automated, microprocessor, motor controller, instruction, algorithm, software, firmware, programmable, wireless, universal serial bus (USB), ethernet, wireless area network (WAN), network enabled.

It will be appreciated that a control module 125 for controlling and recording the various features of the culturing device 100 and of the culture 103 provides for precision, automation, and remote location operation and enables the scaling the number of culturing devices to permit evaluations to be performed in in multiples, in parallel, and sequentially.

It will be appreciated that preserving the composition, mechanical properties, and thermodynamic characteristics of a culture 103 is provided by the present invention of a culturing device 100 for culturing microorganisms from an animal gut. The present disclosure is achieves mixing of the culture 103, extraction of materials from the culture 103 via a conduit 109a and/or 109b, administration of materials to the culture 103 via a conduit, and by using moving means 120 and control module 125 to precisely and accurately prescribe movements and positions of the vessel 101 about an axis of rotation 121 in a way that is non-destructive to the culture 103, and particularly preserves the liquor 104, the headspace 105, and the fibre mat at the interface 106.

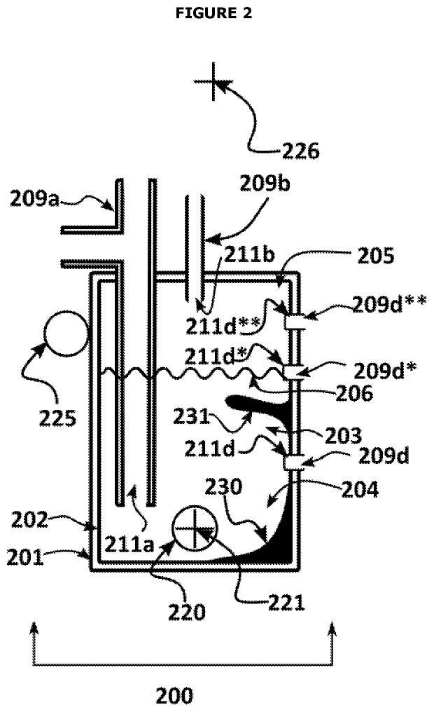

FIG. 2 depicts an example of a culturing device 200 shown in schematic transverse cross-section showing a vessel 201, vessel wall 202, culture 203, liquor 204, headspace 205, interface 207, examples of conduit 209a, 209b, and 209d, examples of moving means, 225 and 225, examples of moving means 220 and 225, examples of axis of rotation 221 and 226, and examples of vessel shape features 230 and 231.

A first exemplary conduit 209a is depicted as fluidically connected to the vessel 201 through the vessel wall 202 and passing through the headspace 205, through the interface 206 and contacting the liquor 204 providing a path for the culture 203 predominantly the liquor 204 through the internal conduit opening 211a. The conduit 209a is predominantly internal to the vessel 201, which has the advantage that the conduit 209a and does not require additional temperature management.

In some examples the conduit 209a can be adjusted or otherwise set so that the internal conduit opening 211a to contact the liquor 202 at one or more prescribed position.

A second exemplary conduit 209b is depicted as fluidically connected to the vessel 201 through the vessel wall 202 and contacting the headspace 205 providing a path for the culture 203, predominantly the headspace 205 through the internal conduit opening 211b.

A third exemplary conduit 209d is depicted as fluidically connected to the vessel 201 through the vessel wall 202 at various positions 209d, 209d*, and 209d** to contact the culture 203, predominantly the liquor 204, the interface 206, or the headspace 205, respectively while the vessel 201 is in the orientation depicted 200. The conduit opening 211d, 211d*, and 211d** of 209d, 209d* and 209d** respectively, provide a path for the culture 203 and are depicted as closed or otherwise sealed with a component that can be temporarily opened and then closed for administering a material and/or extracting a material at a specific part the culture 203, predominantly the liquor 204, the interface 206, or the headspace 205. In some examples the said component can be selected from the group comprising valve, self-sealing injection port, screw port and manifold.

In some examples, at least one conduit 209d can be positioned in the vessel wall 202 to contact the culture 203 and, by specifying parameters of movement and intervals of stillness with the moving means 220 and 225, causes the culture 203 to move within the vessel 201 to a predetermined position so that the liquor 204, or the headspace 205 or the interface 206 can be aligned with said conduit 209d to permit the administration and/or extraction of material at a specific part of the culture 203.

It will be appreciated that a vessel 201 can include at least one conduit 209a, 209b, and 209d and at least one type of conduit 209a, 209b, and 209d.

A first exemplary moving means 220 is depicted as mechanically connected to the vessel 201 via an axis of rotation 220 and acts on the vessel 203 through said mechanical connection. In some examples the said mechanical connection can be selected from the group comprising contact, interference, friction, axle, driveshaft, gear, gearbox, cam, pully and drive belt.

A second exemplary moving means 225 is depicted as mechanically connected to the vessel independent of the axis of rotation 226 and acts directly on the vessel 203 through said mechanical connection. In some examples the said mechanical connection can be selected from the group comprising contact, interference, friction, axle, driveshaft, gear, gearbox, cam, pully and drive belt.

A first exemplary axis of rotation 221 is depicted as inside the profile of the vessel 201.

A second exemplary axis of rotation 226 is depicted as outside the profile of the vessel 201.

In some examples the axis of rotation 221 or 226 can be adjusted.

A first exemplary vessel shape feature 230 is depicted as a curvature of the vessel wall 202. In some examples the said curvature can be selected from the group planer include surface features such as textures or patterning.

A second exemplary vessel shape feature 231 is depicted as a protrusion of the vessel wall 202. In some examples the said protrusion can be selected from the group comprising baffle, channel, wall. In some examples the said protrusion can be rigid or flexible, hinged or elastomeric and include surface features such as textures or patterning.

It will be appreciated that the position of the axis of rotation 221 and 226 and vessel shape features 230 and 231 may assist in optimisation of the mixing and the tumbling of the culture 203 to accurately mimic the mixing and pressure dynamics of fluids and solids within a gut organ in vivo.

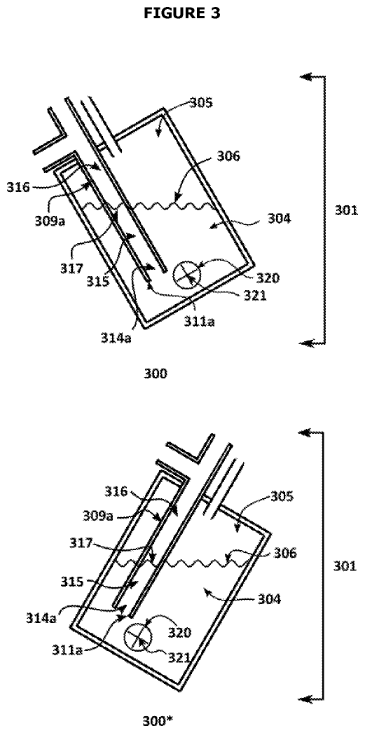

FIG. 3 depicts examples of a culturing device 300 and 300* shown in schematic transverse cross-section showing the vessel 301 in two predetermined positions. In a first predetermined position 300 the moving means 320 has moved the vessel 301 about 30 degrees anticlockwise through a rotational axis 321 from the position shown in FIG. 1 100. In a second predetermined position 300* the moving means 320 has moved the vessel 301 about 30 degrees clockwise through a rotational axis 321 from the position shown in FIG. 1 100, which is about 60 degrees from the position shown in 300.

As depicted in FIG. 3, the moving means 321 moves the vessel 301 from at least one first position 300 to at least one second position 300* and moves at least one portion of the culture 303, predominantly the liquor 304 between the vessel 301 and the conduit 309a to change at least one parameter of said culture.

It will be appreciated that the liquor 304 and the conduit liquor 315 are in fluidic communication via the path 314a at the conduit internal opening 311a and are otherwise exchanged and the results in the transfer of the liquor 304 between the vessel 301 and the conduit 309 and the exchange of conduit liquor 315 between the conduit 309a and the vessel 301.

In some examples the moving of the vessel 301 causes the culture 303 to shift, predominantly the liquor 304 to shift and cause a change in the pressure of the headspace 305.

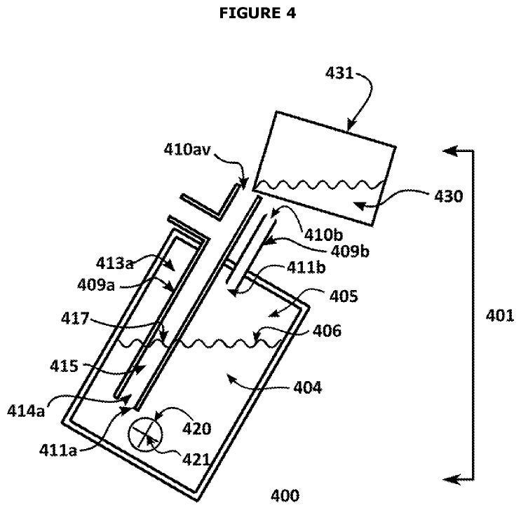

FIG. 4 depicts an example of culturing 400 device shown in schematic transverse cross-section in a predetermined position wherein the vessel 401 has been moved by the moving means 420 about the rotational axis 421 by about 30 degrees clockwise from the position depicted in FIG. 1 100. Also depicted is a material 430 to be administered into the vessel 401 via the conduit 409a from a dispensing instrument 431 proximal to the external conduit opening 410av.

In some examples it may be advantageous to administer a material 430 into the conduit 409a with the vessel 401 in a predetermined position wherein the amount of conduit liquor 415 in the conduit 409a is reduced, which lowers the conduit interface 417 and reduces the hydrostatic impedance of the path 414a thereby assisting in the administration of the material 430 to the vessel 401.

In some examples a material 430 may be administered to the vessel 401 through the conduit 409a using a dispensing instrument 431 proximal to the external conduit opening 410av which may be partially or fully inserted into the external conduit opening 410av to permit the material 430 to enter the conduit 409a without appreciable loss of material 430.

In some examples the dispensing instrument 431 may include a standard apparatus for administering liquid, particulate, or solid material 430 selected from the group comprising beaker, pipette, jug, funnel, hopper, pump, tube, syringe.

In some examples a material 430 may be administered in an automated or semi-automated fashion using a machine fitted with a dispensing instrument 431 to dispense a predetermined material 430, a predetermined amount of material 430, and at a predetermined time. The machine may be managed or be in communication with the control module depicted in FIG. 1 125 so that the administration of a material 430 can be synchronised with the position of the vessel 401 to ensure correct alignment of the dispensing instrument 431 with the external conduit opening 410av.

The conduit 409a may also incorporate means to assist the administration of material 430 into the vessel 401 to reduce impedance in the conduit 409a and minimise the chances of blockage in the path 414a selected from the group comprising: plunger, feed screw, auger, vibrator, conveyer, valve, tap, manual, motorised, automated.

It will be appreciated that the administration of material 430 to the vessel 401 via the conduit 409a will cause the content of the vessel 401 to be changed.

In some examples the administration of material 430 enables the introduction of material 430 to the culture 403. In some examples the administration of material 430 to the vessel 401 may change the properties of the culture 403 comprising changes to the properties of the liquor 404 and changes to the properties of the headspace 405 selected from the group comprising volume, pressure, composition, and temperature.

In some examples when the conduit 409b is appreciably unobstructed the administration of material 430 to the vessel 401 may cause the level of the conduit interface 417 to rise, which conduit liquor 415 in fluidic communication with the liquor 404 changes the volume of the liquor 404 and the interface 406 to equilibrate and become level with the conduit interface 417. However, if the conduit 409b providing a path 414b for the headspace 405 is predominantly occluded and the vessel 401 is suitably gastight the interface 406 will not equilibrate to level with the conduit interface 417 and the pressure of the headspace 405 will be increased proportional to the head created by the rise in the level of the interface 417 in the conduit 409a. In some examples the conduit 409b providing the path 414b for headspace 405 can be adjusted to change pressure and/or volume of the headspace 405 and or volume of the liquor 404 using pressure adjustment means selected from the group comprising valve, tap pressure regulator airlock.

In some examples a material 430 may be any material 430 to be administered to the vessel 401 for establishing the culture 403, maintaining the culture 403, testing the culture 403, or for testing a response of the culture 403 to an administered material 430. The administration of material 430 can occur in one or more steps separated by one or more intervals of time with material 430 selected from the group comprising:
(i) material from foregut organs of live or dead animals selected from the group comprising the suborder Ruminantia (ruminants) including domesticated animals, cattle, goats, sheep, bison, buffalo, yaks, deer, antelope, the interior and contents of the pseudo-rumen of live animals from the suborder Tylopoda (pseudo-ruminants) including camels, alpacas and lamas, rumen, reticulo-rumen, caecum, intestine, colon:
(ii) material from foregut models including, mechanical rumens, artificial rumens, rumen simulation technique (RUSITEC), and bioreactors;
(iii) a microorganism including cultured or naturally sourced microorganisms selected from the group comprising archaea, methanogens, bacteria, mycobacterium, fungi, viruses, and protozoa;
(iv) organic and inorganic material including fluids, liquids and gases, dissolved gas, atmospheric gas, purified or refined gases, and acidity regulators selected from the group comprising hydrogen (H), molecular hydrogen (H₂), ionic hydrogen (H⁺), oxygen (O), molecular oxygen (O₂), carbon dioxide (CO₂), nitrogen (N), ammonia (NH₃), ammonium (NH₄⁺), molecular nitrogen (N₂), hydrogen sulfide (H₂S), methane (CH₄), water, mineral salts, ions, bicarbonate, buffer;
(v) a biochemical selected from the group comprising amino acids, peptides, proteins, nucleic acids, carbohydrates, fatty acids, volatile fatty acids, volatile fatty acid, acetic acid, acetate, propionic acid, propionate, butyric acid, butyrate, isobutyric acid, isobutyrate, valeric acid, valerate, heptanoic acid, heptanoate, co-factors, flavin co-factors, co-factor F420, polyphenols;
(vi) a feed material or feed supplement selected from the group comprising, forage, pasture, grass, Gramineae, legume, Leguminosae, clover, lucerne, fodder, preserved, straw, hay, silage, baleage, brassica, herb, chicory, plantain, grains, oats, barley, maize;
(vii) a methane reducing treatment, supplement, or feed supplement selected from the group comprising drug, inhibitor, methanogenesis inhibitor, organohalogen, haloform, chloroform, bromoform, iodoform, seaweed, Asparagopsis sp., Asparagopsis extract, Asparagopsis matter, alliin, iso-alliin, allicin, Mootral™, garlic extract, garlic matter, 3-Nitrooxypropanol (3-NOP), Bovaer™; and
(viii) a device selected from the group comprising sensors, bolus, and electronic bolus.

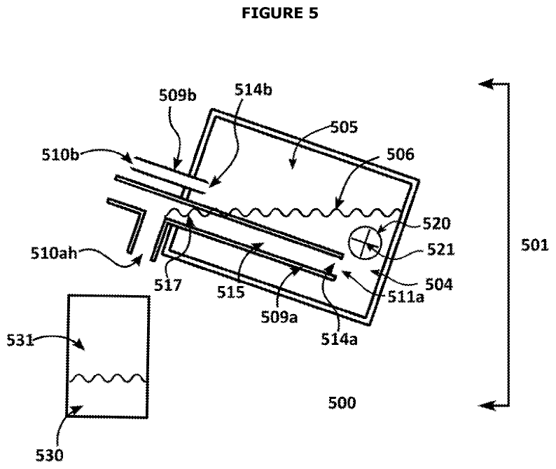

FIG. 5 depicts an example of culturing 500 device shown in schematic transverse cross-section in a predetermined position wherein the vessel 501 has been moved by the moving means 520 about the rotational axis 521 by about an angle of 71 degrees anticlockwise from the position depicted in FIG. 1 100.

Also depicted is a material 530 extracted from the vessel 501 via the conduit 509a using an extraction instrument 531 proximal to the external conduit opening 510ah.

In some examples the material 530 is the conduit liquor 515 and by fluidic connection through the path 514b the liquor 504.

In some examples it may be advantageous to extract the material 530 via the conduit 509a by moving to a predetermined position which raises the conduit liquor 515 in the conduit 509a to a threshold level thereby causing in the conduit interface 517 to breach the external conduit opening 510ah and cause the conduit liquor 515 to be poured and/or decanted from the external conduit opening 510ah into a collection instrument 531.

In some examples various increments of position can be prescribed to cause the predetermined pouring and/or decanting of conduit liquor 515 to cause the retention of predetermined volumes of liquor 504 in the vessel 501.

In some examples a material may be extracted from the vessel 501 via the conduit 509a using an extraction instrument 530 selected from the group comprising beaker, pipette, syringe, needle, jug, funnel.

In some examples the position is predetermined to pour and/or decant a prescribed amount of material 530 from the conduit 509a or to retain a prescribed amount of culture 503 predominantly liquor 504 in the vessel 501

In some examples the position of the vessel 501 is adjusted to a predetermined position to cause the vessel liquor 504 to enter the conduit 509a and be poured and/or decanted from the external conduit opening 510ah and the material 530 may be collected in a collection instrument 531. In some examples a material 530 may be extracted in an automated or semi-automated fashion using a machine fitted with an extraction instrument 531 to extract a material 531 of a predetermined amount, at a predetermined time for example a fraction collector or autosampler. The machine may be managed or be in communication with the control module depicted in FIG. 1 125 so that the extraction of a material 530c can be synchronised with the position of the vessel 501 to ensure correct alignment of the extraction instrument 531 with the external conduit opening 510ah.

In some examples the conduit 509a may also incorporate means to assist the extraction of material 530 from the vessel 501 to reduce impedance in the conduit 509a and minimise the chances of blockage in the path 514a selected from the group comprising: plunger, feed screw, auger, vibrator, conveyer, valve, tap, manual, motorised, automated.

It will be appreciated that the extraction of material 530 from the vessel 501 via the conduit 509a will cause the content of the vessel 501 to be changed.

In some examples the extraction of material 530 enables the extraction of predominantly the liquor 504. In some examples the extraction of material 530 from the vessel 501 may change the properties of the culture 503 comprising changes to the properties of the liquor 504 and changes to the properties of the headspace 505 selected from the group comprising volume, pressure, composition, and temperature.

In some examples when the conduit 509b is appreciably unobstructed the extraction of material 530 from the vessel 501 may cause the level of the conduit interface 517 to rise, which conduit liquor 515 in fluidic communication with the liquor 504 changes the volume of the liquor 504 and the interface 506 to equilibrate and become level with the conduit interface 517. However, if the conduit 509b providing a path 514b for the headspace 505 is predominantly occluded and the vessel 501 is suitably gastight the interface 506 will not equilibrate to level with the conduit interface 517 and the pressure of the headspace 505 will be adjusted proportional to the head of pressure created by the rise in the level of the interface 517 in the conduit 509a. In some examples the conduit 509b providing the path 514b for headspace 505 can be adjusted to modulate pressure and/or volume of the headspace 505 and or volume of the liquor 504 using pressure adjustment means selected from the group comprising valve, tap pressure regulator airlock.

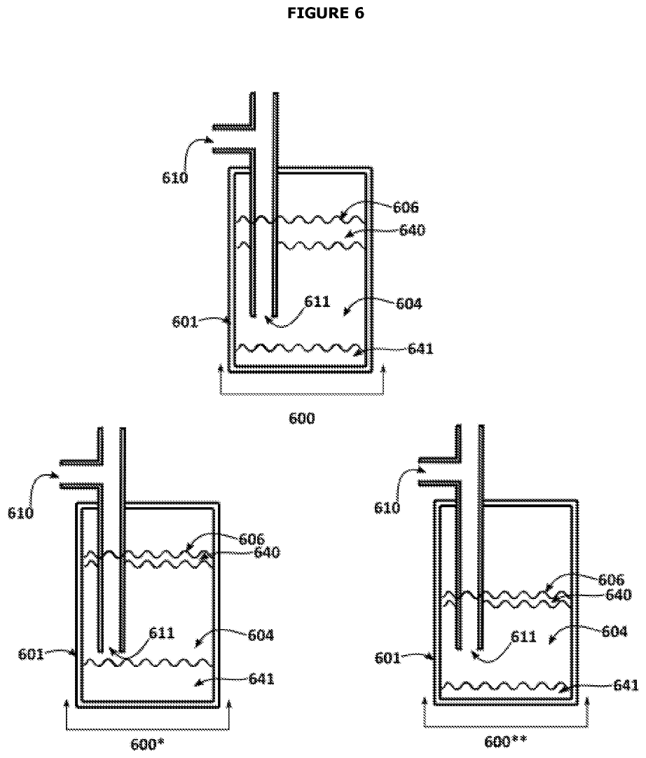

FIG. 6 depicts examples of a culturing device 600 shown in schematic transverse cross-section showing the vessel 601 in one predetermined position and changes in the composition of the liquor 604 due to the administration of feed material 600 as previously described herein, due to the incubation of the culture 600* at prescribed intervals of movement and stillness as previously described herein, and due to the extraction of liquor 600** as previously described herein.

In some examples the liquor 604 can include a fibre mat 640, which exists in the liquor and proximal to the interface 606 and is comprised of substantially buoyant and/or undigested material. In some examples the liquor 604 can additionally or separately include a sediment 641 which exists in the liquor 604 and is proximal to the base of the vessel 601 and is comprised of substantially dense and/or digested material.

It will be appreciated that when a feed material has been administered to a culturing device 600 the proportion of fibre mat 640 will be at least in part due to the composition of the feed material, but that after some period of incubation 600*, for example 24 hours, the fibre mat 640 decreases at least in part due to digestion of the fibre mat 640 by the microorganisms of the liquor 604. In some examples the digestion causes the formation and accumulation of sediment as depicted in 600*.

In some examples the pouring and/or decanting of liquor 604 by causing the vessel to move to a prescribed position for a prescribed interval of stillness will cause the liquor 602 to enter the conduit 609 via the internal conduit opening 611 and then exit the conduit via the external conduit opening 610 and so cause the amount of liquor 604 to be reduced inside the vessel 601 as depicted in 600**.

In some examples prescribing the position of the internal conduit opening 611 combined with the prescribed movement and prescribed interval of stillness will predictably control the composition of the liquor 604 decanted and the change in the composition of the liquor 604 retained with respect to the proportion of fibre mat 640 and/or the sediment 641.

It will be appreciated that an important feature of the disclosure is the preservation of the liquor 604 with respect to the fibre mat 640 and/or the sediment 641 due to the prescribed movement and prescribed interval of stillness applied to the vessel 601 by the culturing device 600, 600*, and 600**.

The culturing device disclosed herein and illustrated by way of example provides a substantially closed vessel for the culturing of microorganisms from the animal gut, particularly adapted for culturing microorganisms from the ruminant animal foregut. The culture is predominantly located in the vessel of the culturing device. The culture in the vessel is in hydraulic contact with culture in the conduit and the conduit may be open, partially open, or closed.

The moving of the vessel when performed as programmed sequences of predetermined movements and intervals of stillness permits the culture in the vessel to exchange with the conduit lumen via hydraulic contact in a precisely controlled fashion to change one or more parameters of the culture.

The culturing device can be operated with great precision and is amenable to machine automation, particularly computer programmable automation, which provides the user with utilities that makes the culturing device inexpensive to run, simple to operate, and highly reliable for methods comprising:
(i) the administration of materials to the device including inoculation of the vessel with a culture of microorganisms from an animal particularly the microorganisms from the foregut organs of a ruminant or pseudo-ruminant animal comprising archaea, bacteria, fungi, viruses, and protozoa;
(ii) the administration of materials to the culture including feeding the culture with food, nutrients, and supplements;
(iii) the administration of materials to the culture including medicaments, microorganism inhibitors, and microorganism enhancers;
(iv) the maintaining the culture at temperatures and pressures predetermined as most efficient for culturing microorganisms from an animal particularly the foregut organs of a ruminant or pseudo-ruminant animal;
(v) the preservation of important structural features of the culture of microorganisms from an animal, particularly the foregut of a ruminant animal including the liquor, the interface, the fibre mat, the headspace, the gas cap; and
(vi) the extraction of materials from the culture including removing samples of the liquor and/or samples of the headspace and/or samples of the interface for testing, measurement, sample extraction, analysis, and disposal.

The culturing device provides a useful, inexpensive, and scalable alternative to live animal testing for the development and validation of treatments, feeds, and supplements to reduce methane gas emissions from ruminant and pseudo-ruminant animals, particularly farmed animals.

### Example 2: Development of Prototype Devices

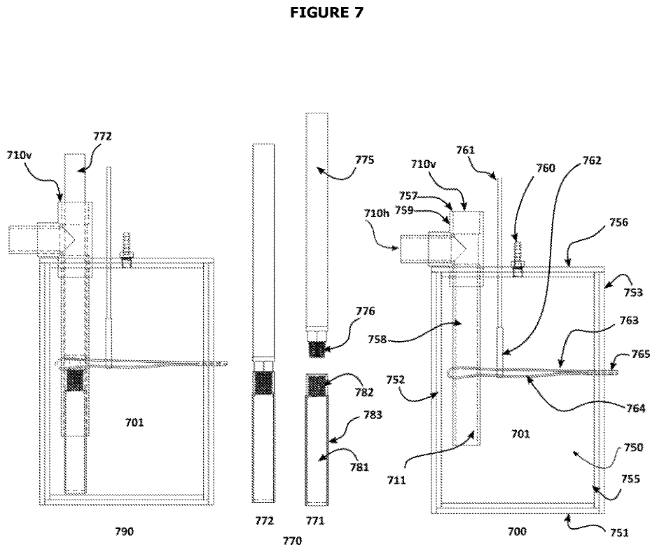

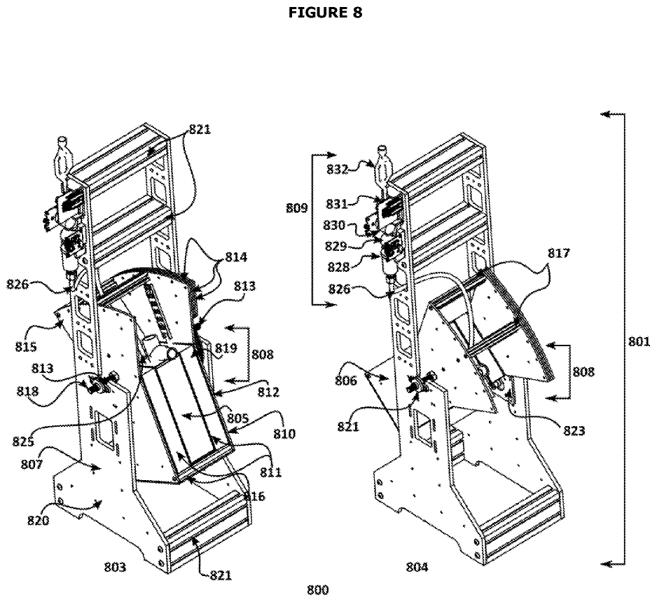

A prototype culturing device (i.e. to satisfy scale-up requirements) in accordance with the present invention was constructed as described below with reference to FIGS. 7 and 8.

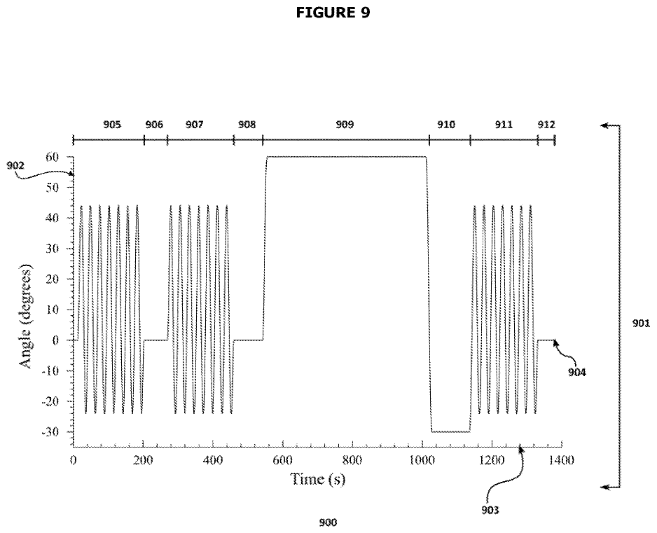

These devices were then tested to measure parameters such as (e.g.) movement and pressure/temperature fluctuations under culture conditions as a function of time (i.e.) refer to description of FIGS. 9-12 which follows.

FIG. 7 depicts a computer aided design (CAD) showing in right hand projection an example of a manufactured vessel 700 incorporating a conduit and a dissolved hydrogen sensor, a VFA sink apparatus 770 and vessel with VFA sink apparatus installed 790. The manufactured vessel 700 comprises a vessel 701 with internal dimensions 60 mm×152 mm×230 mm to provide an enclosed volume 750 of about 2 L, that when in use typically contained a liquor volume of about 1.5 L and a headspace volume of about 0.5 L comprising:

The vessel 701 had a body 750 fabricated from five pieces of acrylic (polymethylmethacrylate) sheet 6 mm to 10 mm thick bonded at their edges with dichloromethane to form an open box with faces comprising the base 751, front 752, back 753, left 754, and right 755. A lid 756 for the body 750 was fabricated from PVC and bonded to the open top of the body 750 with silicone sealant. Inserted through the lid 756 were components comprising the following components.
(i) A conduit 757 comprising a 20 mm inner diameter PVC tube 758 of length 160 mm bonded at one end to the primary axis of a plain 20 mm PVC tee 759 mounted through the lid 756 proximal to the front face 752 of the vessel 701 with two external conduit openings 710h and 710v to atmosphere and the internal conduit opening 711 positioned about 60 mm above the internal surface of the vessel base 751. When in use the conduit inside the vessel extended through the liquor-headspace interface and provided a path from outside of the vessel to inside the vessel wherein the internal conduit opening 711 was positioned under the interface and contacting the vessel liquor. The conduit contained and was in hydraulic communication with the vessel liquor and when moved, the height of the liquor inside the conduit tube equilibrated with the height of the liquor level in the vessel, the external opening opened the conduit tube provided a path to the vessel liquor, permitted the vessel to be inoculated with a culture, the of the vessel liquor to be amended by the administration of materials including: food, water, buffer, supplements, inoculants, and chemicals, through the conduit tube, and by tilting the vessel a portion of the liquor to be poured and/or decanted from the vessel and so extracted.
(ii) A second conduit comprising a stainless steel 6 mm hose barb connector 760 with a ⅛th BSP male thread screwed into a ⅛th BSP threaded hole the centre of the lid 756 and glued with epoxy to ensure a gas-tight seal. When in use a flexible PVC tube of length about 1 m×6 mm inner diameter) fitted to the hose barb connector 760 provided a path for emitted gases from the vessel headspace to a gas handling apparatus including a pressure adjustment means comprising a pressure sensor and a fermentation airlock.
(iii) A temperature sensor 761 with stainless steel waterproof probe 762 (6 mm×50 mm) encasing a DS18B20 programmable resolution 1-wire digital thermometer. The temperature sensor probe 761 was inserted through a gas-tight aperture in the lid 756 so that the sensor element 762 was positioned at about the midpoint of the vessel 702 so that when in use was in contact with the culture liquor.

The vessel 701 had passing through a hole in its back face 753 a sensor 763 configured to contact the culture and monitor dissolved hydrogen. The sensor 763 comprised a 400 mm loop of perfluoroalkoxy (PFA) polymer tubing 764 of cross-sectional dimensions 1.59 mm outer diameter, 1 mm inner diameter, with each end of the tubing 764 passed through a gas tight gasket 766 comprising a piece of aluminium tubing 25 mm 4 mm outer diameter. The aluminium gasket 766 was glued into a receiving hole in the back face 753 at about the mid-point of the vessel 702 and potted with epoxy such that both ends of the tubing were open to atmosphere. When in use the loop 764 was immersed in the liquor inside the vessel 701 and the open ends of the tubing were piped to the hydrogen inlets of a hydrogen fuel cell (IronSuperman, with a 25 mm² active area and a generating voltage range 0 V-0.9 V). The fuel cell's oxygen inlets were left open to air and the analog voltage generated at its electrodes was monitored with a multimeter and recorded using an analog to digital converter and a computer connected to a system control module. A positive voltage indicated dH₂ in the liquor at a level greater than atmospheric H₂ had diffused into the lumen of the tubing 764 and contacted the hydrogen electrode of the fuel cell. A negative signal indicated a dH₂ level in the liquor less than atmospheric H₂.

Also depicted in FIG. 7 is an example VFA sink apparatus 770 configured to contain an ion exchange resin to absorb VFAs. The VFA sink apparatus comprised a two-part cartridge 770 and is shown in a disassembled form 771 and an assembled form 772. The cartridge 770 was manufactured by fuse filament deposition printing of polyethylene terephthalate modified with glycol (PETG). The cartridge 770 comprised a cartridge handle 775 (20 mm outer diameter×200 mm) incorporating a male M16 thread adapter 776 and a cartridge proper 780 (20 mm outer diameter×130 mm) incorporating a female M16 thread adapter 788 to allow the cartridge handle 775 to be joined to the cartridge proper 780. The cartridge proper 780 had an internal cavity 781 (19 mm inner diameter×100 mm) with cavity walls 783 that were 0.5 mm thick. In use the cavity 781 was filled with a sample of microporous ion exchange resin particles with a particle size of 500-700 μm and the cavity 781 closed, thereby encapsulating the resin, when the cartridge handle 775 was joined to the cartridge proper 780 as depicted in the assembled form 772. The cavity walls 783 were permeable to aqueous liquids but retained the resin particles to allow the resin particles to be bathed with aqueous fluid while being retained in the cavity 781.

The assembled cartridge 772, containing ion exchange resin in its cavity 781, provided a VFA sink apparatus that was configured to be inserted into the conduit 758 of a vessel 701 via the external conduit opening 710v as depicted 790. The construction of the VFA sink apparatus 770 with a handle 775 (total length of about 330 mm) provided a convenient means to insert ion exchange material into the liquor of a functioning vessel 701 containing a culture of microorganisms. Furthermore, the VFA sink apparatus 770 can be periodically removed and adsorbed materials eluted and subjected to analysis and the VFA sink apparatus recycled for repeat usage.

In use the cavity 781 was filled with a weak anion exchange resin comprising 28 mL of Amberlite IRA67 which has a matrix of cross-linked acrylic acid and a polyamine functional group and known to absorb the VFA acetate from fermentation fluids with a high binding efficiency. See for example: Karekar S C, Srinivas K, Ahring B K. Batch screening of weak base ion exchange resins for optimized extraction of acetic acid under fermentation conditions. Chemical Engineering Journal Advances. 2022 Aug. 15; 11:100337.

After inoculation and or feeding the vessel 701 of the culturing device, VFA sink apparatus was inserted into the vessel and retained there for a user defined interval of time.

FIG. 8 is a CAD depiction 800 of an example manufactured culturing device 801 shown in isometric projection in two configurations of operation 803, 804. The left configuration 803 has an identical composition to the right configuration 804 and features marked on either or both configuration 803, 804 pertain to the key mechanical features of the culturing device 801 and referred to generally as the culturing device 801 unless referring to a specific configuration 803 or 804.

The culturing device 801 was made from sub-assemblies comprising vessel 805, cradle 806, chassis 807, drive train 808, and gas line 809.

The vessel 805 is identical to the vessel 702 explained in FIG. 7 700 and the accompanying description.

The cradle 806 was a receptacle for the vessel 805 to hold the vessel 805 therein by interference, which holding was augmented with a zip tie. The cradle 806 comprises a cradle body 810, thermal insulation 811, heater elements 812, an axle 813, and counterweights 814.

The cradle body 810 comprised two symmetrical cradle sides 815 fabricated from 5 mm aluminium sheet that were bolted to a base plate 816 and chassis spacers 817 to form a rigid unit. The base plate 816 was fabricated from 10 mm thick mild steel and the chassis spacers 817 were fabricated from 20 mm×20 mm extruded aluminium t-slot. The profile of each cradle side 815 was designed to accommodate the profile of the vessel 805 and includes an extension to permit the addition of separate counterweights 814. To the inner surface of each cradle side 815 was bonded 40 mm thick insulating foam block 811 cut to fit the profile of the vessel 805 and the cradle body 810. To the inside surface of each foam block 811 a cavity was recessed to neatly house a silicon heater mat 812 (30 W, 150×200 mm, 12 V dc), which was held in place by a 2 mm thick aluminium plate 812 cut to the same profile as the foam block 811. In operation the heater mat 812 was insulated on its back face by the foam block 811 and its front face evenly heated the inside aluminium plate 812 in contact with the vessel 805.

The cradle 806 included on each cradle side 815 a cradle axle 813 positioned externally to provide an axis of rotation about the top centre of the cradle 806 and the vessel 805 held therein.

On the end of one axle 813 was attached a solid state MPU-6050 GY-521 Sensor Module 3 Axis Accelerometer Gyroscope 16 Bit AD Converter 818 connected to a bespoke electronic circuit board to control the operation of and facilitate the communication of instructions and data for absolute position encoding of the cradle 806 and the vessel 805 held therein.

On one cradle side 815 was fixed a bespoke 3D-printed GT2 toothed segment pulley 819 of radius 85.4 mm position co-axial with the axis of rotation.

The cradle 806 included counterweights 814 fabricated from 5 mm thick steel plate that could be bolted to at least one cradle side 815 to adjust the centre of gravity of the cradle 806 and the vessel 805 held therein, by adding or removing counterweights.

The chassis 807 comprised two symmetrical chassis sides 820 fabricated from 10 mm thick mild steel that were bolted to chassis spacers 821 to form a rigid unit. The chassis spacers 821 were fabricated from 40 mm×80 mm extruded aluminium t-slot. The profile of each chassis side 820 was designed to accommodate the attachment of features including a bearing 822 for the cradle axle 813, a stepper motor 823 for the drive train 808, and a gas line 809.

The drive train 808 was a synchronous toothed belt and pulley system with a transmission ratio of 14:1 comprising a NEMA 23 stepper motor 823 fitted with a 20-2GT-10 timing pulley, coupled via a 10 mm wide GT2 belt fixed to the segment pulley so that when in use the stepper motor 823 could be controlled to adjust the movement of the cradle 806 and the vessel 805 held therein about the axis of rotation specified by the location of the cradle axle 813 and the bearing 821.

In the operation of the drive train 808, the stepper motor 823 caused the rotational movement of the cradle 806 and the vessel 805 held therein about the rotational axis of the cradle 806. The direction of the movement can cause the cradle 806 to rotate anticlockwise as depicted in configuration 803, where the cradle 806 and the vessel 805 held therein rotated −25 degrees from a position where the vessel 805 was true vertical. Alternatively, the direction of the movement can cause the cradle 806 to rotate clockwise as depicted in configuration 804, where the cradle 806 and the vessel 805 held therein rotated 45 degrees from a position where the vessel 805 was true vertical.

The selection of the stepper motor 823 and the choice of counterweights 814 permits the cradle 806 and the vessel 805 held therein to be held at any angle+90 degrees from a position where the vessel 805 was true vertical for a prescribed interval of time.

The gas line 809 mounted onto the chassis side 820 was a linear configuration of components fluidically connected to the vessel hose barb 825 comprising:
(i) a flexible PVC hose 826 with a 6 mm inner diameter×1.2 m, long enough to accommodate the full range of movement of the cradle 806 and the vessel 805 held therein, connected via a hose barb to;
(ii) an inline water trap 828 (Blackridge), connected via a stainless-steel thread hex nipple union to;
(iii) one CH₄ sensor 829 comprising an AMPHENOL SGX SENSORTECH JAS767906AA Flow Hood Assembly containing an AMPHENOL SGX SENSORTECH CH4 Sensor type INIR-ME 100% with a bespoke electronic circuit board to control the operation of and facilitate the communication of instructions and data, and connected via a stainless-steel thread hex nipple union to;
(iv) one CO₂ sensor an AMPHENOL SGX 830 comprising SENSORTECH JAS767906AA Flow Hood Assembly containing an AMPHENOL SGX SENSORTECH CO2 sensor type INIR-CD 100% with a bespoke electronic circuit board to control the operation of and facilitate the communication of instructions and data and, connected via a stainless steel 6 mm hose barb to;
(v) a flexible PVC hose 6 mm inner diameter×50 mm connected to
(vi) one polyethylene three-way tee joint hose connector (6 mm) with one branch dedicated to:
   (a) a digital pressure sensor 831 with a bespoke electronic circuit board to control the operation of and facilitate the communication of instructions and data, and one branch connected by an interference fit to
   (b) a fermentation twin bubble airlock 832 loaded with 1 ml of paraffin oil to regulate the internal pressure of the gas line 809 and so the vessel 805.

Thereafter gases escaping the fermentation airlock 832 were captured by a non-contacting shroud with a slight negative pressure applied.

The manufactured culturing device 801 disclosed herein was controlled by a module that utilised and permitted the communication of instructions and data to and from the sensors, actuators, and regulators of the culturing device.

The control module enabled the automation and precision operation of at least one culturing device and permitted the scaled operation of more than one culturing device.

The control module comprised bespoke firmware and software applications that were developed using standard tools and methods. The control module enabled the execution of user-configured instructions via the internet and WiFi protocols on a Raspberry Pi microcomputer interfaced via USB to bespoke electronic circuit boards configured with Arduino MKR Zero microcontrollers to facilitate the modularised communication of instructions and data to and from the sensors, actuators, and regulators of the culturing device.

In operation of the manufactured culturing device 801 disclosed herein, a software application was developed and run on a computer, which allowed a user to routinely and easily specify parameters including:
(i) position, which was any angle of the vessel from 0 to #180 degrees;
(ii) set Zero point, which was the manual calibration of the true vertical position of the vessel normally set with a spirit level and thereafter automatically reset based on readings from the 3 Axis Accelerometer;
(iii) forward Angle, which was any prescribed angle of the vessel from 0 to +90 degrees;
(iv) reverse Angle, which was any prescribed angle of the vessel from 0 to −90 degrees;
(v) number of Cycles, which was the number of repetitions between any prescribed Forward Angle and Reverse Angle;
(vi) pause, which was the interval of stillness between cycles at the Forward Angle and/or the Reverse Angle;
(vii) acceleration, which is the angular acceleration of the stepper motor to a maximum angular velocity of the stepper motor;
(viii) velocity, which is the maximum angular velocity of the stepper motor; and
(ix) temperature, which is the setpoint temperature inside the vessel.

The recording of data from the peripheral sensors, actuators, and regulators of the culturing device was performed autonomously and communicated by internet to a structured query language (SQL) database where the data was accessed by the application Navicat for MySQL by CyberTech Ltd., and a bespoke software application developed for customised analysis and presentation of data from the culturing device 801.

Thus, the user was able to prescribe the various control parameters and record the various control and sensor outputs of the culturing device with a high degree of precision, integrity, and automation.

In some examples, the user was able to prescribe the Forward Angle to pour and/or decant the liquor contents of the vessel and retain a known and reproducible volume of liquor in the vessel.

In some examples, the user was able to prescribe various patterns of Forward Angle, Reverse Angle, and Pause, for a specific Number of Cycles, or alternatively indefinitely until terminated by the user and record synchronous measurements from the sensors and perform asynchronous measurements on decanted samples extracted from the liquor. This had the advantage of enabling the user to test various patterns vessel movements and determine by measurements from the sensors the effects of movement on the maintenance and growth of the microorganism culture in the liquor and the production dH₂ and CH₄.

In some examples the user was able to test the effect of different feed materials including cut and dried feed, fresh feed, ryegrass and lucerne, the frequency of feeding including once every 12 hours, 24 hours or 36 hours, the amount of feeding and record synchronous measurements from the sensors and perform asynchronous measurements on decanted samples extracted from the liquor. This has the advantage of enabling the user to test various feeding regimes and feed types and determine by measurements from the sensors the effects of feed on the maintenance and growth of the microorganism culture in the liquor and the production dH₂ and CH₄.

In some examples the user was able to test the effect of different inoculation cultures from different organs of one animal including the rumen and the caecum from a sheep, from different animals including from sheep and cow, from faecal sources including from sheep and cow, the amount of inoculum, the preparation of the inoculum and record synchronous measurements from the sensors and perform asynchronous measurements on decanted samples extracted from the liquor. This has the advantage of enabling the user to test various inoculation regimes and inoculation types and determine by measurements from the sensors the effects of inoculum on the maintenance and growth of the microorganism culture in the liquor and the production dH₂ and CH₄.

In some examples the user was able to test the effect of different feed additives, the type of supplement including glycerol, canola oil and ground oats, the amount of feed supplement, and the frequency of feeding the feed supplement, and record synchronous measurements from the sensors and perform asynchronous measurements on decanted samples extracted from the liquor. This has the advantage of enabling the user to test various feeding additives and feed types and determine by measurements from the sensors the effects of feed supplement on the maintenance and growth of the microorganism culture in the liquor and the production dH₂ and CH₄.

In some examples the user was able to test the effect of a methanogenesis inhibitor including CHBr3, the amount of methanogenesis inhibitor, the frequency of administration of a methanogenesis inhibitor, and the recovery post methanogenesis inhibitor administration, and record synchronous measurements from the sensors and perform asynchronous measurements on decanted samples extracted from the liquor. This has the advantage of enabling the user to test various methanogenesis inhibitor and determine by measurements from the sensors the effects of methanogenesis inhibitor on the maintenance and growth of the microorganism culture in the liquor and the production dH₂ and CH₄.

It will be appreciated that many variations of the culturing device disclosed herein have been developed and evaluated to arrive at the examples disclosed herein. In some examples the axis of rotation was located towards the bottom of the vessel. In some examples the drive train comprised a stepper motor configured with a planetary gearbox. In some examples the vessel was fabricated from opaque PVC. In some examples the conduit was of the type explained in FIG. 2 209c and the accompanying description. In some examples the conduit was closed or partially closed during the non-pouring/non-decanting, non-administration applications of the culturing device. In some examples the gas sensors were positioned posterior to the fermentation airlock. In some examples the gas line lacked a H2S scrubber and/or water trap. In some examples the gas line incorporated a disc micropump from Ion Science Royston, SG8 7SL, United Kingdom. In some examples a 124 L vessel has been constructed representing a full-scale model of a large cow rumen volume.

It will be appreciated that many variations of the culturing device disclosed herein have been envisaged. The culturing device can be scaled to any size appropriate for the purpose. For example, the average sheep has a rumen of volume 6 L. In some examples the culturing device could use a vessel of volume 2 L thereby representing a ⅓rd scale of the average sheep rumen volume. Alternatively, a 6 L vessel could be constructed representing a full-scale model of the sheep rumen volume. In another example the average cow has a rumen volume of about 80 L. A 80 L vessel could be constructed representing a full-scale model of the cow rumen volume.

A culturing device as disclosed herein was used as described below with reference to FIGS. 9, 10, and 11.

FIG. 9 shows an example recording 900 of prescribed movement comprising a graph 901 of the angle on the y-axis 902 in degrees versus time on the x-axis 903 in seconds for the plot 904 of the angle of the vessel of the culturing device containing liquor about an axis of rotation. The angle shown on the y-axis 902 is the angle prescribed by the programmed steps of the stepper motor driver and based on the position of the vessel from a true vertical orientation at 0 degrees set and monitored with a digital accelerometer. Thus, the prescribed size and direction of movement of the vessel is reflected in the amplitude and sign of the angle rotation from 0 degrees. The slope of the plot 904 between any two angles is dependent on the acceleration and velocity of the movement.

At the top of the graph 901 are bars indicating extended intervals of movement 905, 907, and 911, and extended intervals of stillness 906, 908, 909, 910, and 912. In one interval of movement 907, seven cycles of prescribed movement of the vessel between-24 degrees and 44 degrees that was preceded by an interval of stillness 906 at an angle of 0 degrees and followed by an interval of stillness 908 at an angle of 0 degrees. It will be appreciated that there are also momentary intervals of stillness as the vessel stops and changes direction in such cycles of movement. The concatenation of various cycles of movement and stillness can be used for the semicontinuous mixing of the liquor inside the vessel as explained by FIG. 3 and the accompanying description. The parameters of movement of the vessel can be adjusted by prescribing the number, frequency, amplitude direction, acceleration, and velocity by programming the stepper motor driver to move in ⅛th micro steps at velocities up to a maximum of 16 degrees/s and accelerations up to a maximum of 2 degrees/s².

Indicated in the graph is the interval of stillness 909 showing the holding of the vessel at 60 degrees from true vertical for a period of 480 seconds, which provides an example of decanting the vessel for the extraction of liquor as explained by FIG. 5 and the accompanying description. This is followed by an interval of stillness 910 showing the holding of the vessel at −30 degrees from true vertical for 120 seconds, which provides an example of administration liquor as explained by FIG. 4 and the accompanying description.

FIG. 10 shows an example recording 1000 of the effect on headspace pressure due to prescribed movement of the vessel comprising a graph 1001 of measured pressure on the upper y-axis 1002 in Pascals and the prescribed movement and on the lower y-axis 1003 in degrees versus time on the x-axis 1004 in seconds. The plot 1005 of pressure is the pressure on the headspace recorded by an electronic pressure sensor. The plot 1006 shown in degrees is the angle of the culturing device vessel containing liquor about an axis of rotation as explained by FIG. 9 and the accompanying description.

At the top of the graph 1001 are bars indicating intervals of stillness 1007 and intervals of movement 1008 reflected in the plot 1006 of movement. The intervals of movement 1008 comprise six cycles of prescribed movement of the vessel between −24 degrees and 44 degrees that was preceded and followed by an interval of stillness 1007 at an angle of 0 degrees.

As can be seen from the graph 1001, the movement of the vessel causes the pressure inside the vessel containing the liquor and the headspace to change as reflected in the plot 1005 of the measured pressure. This indicates that moving of the vessel causes movement and thus mixing of the liquor and headspace fluids of the culture, which was confirmed by visual inspection of the contents of the vessel through the transparent portions of the vessel.

It will be appreciated that measurement of the pressure and the change in pressure is useful to determine the flux of emitted gas from the culture and to measure, control, and release of emitted gas from the headspace.

To generate the pattern of movement generating intervals of movement 1008 and intervals of stillness 1007, an algorithm implemented by the control software provided machine instructions for predetermined movements and intervals of stillness comprising:

```
Start
    Repeat until paused
    Set the vessel to 0 degrees (true vertical)
      Repeat 6 times
        Move to + 44 degrees, pause for 0 seconds
        Move to − 24 degrees, pause for 0 seconds
    Move to 0 degrees
    Pause 60 seconds
    Repeat
  End
```

It will be appreciated that any regular pattern or irregular arrangement of movement can be prescribed by the culturing device and permit the optimisation of the mixing of the culture and the preservation of the structural features of the culture that is sympathetic to movement of the animal gut contents.

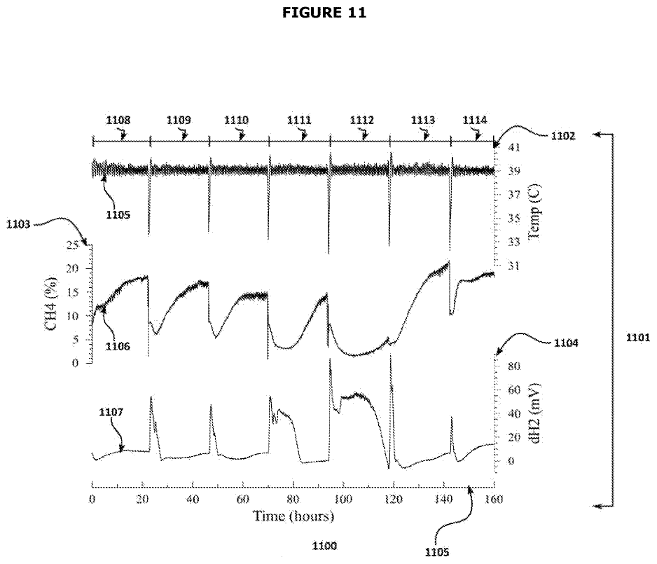

FIG. 11 shows an example recording 1100 of the temperature, CH₄, and dH₂ due to the experimental administration of feed material and the methanogenesis inhibitor CHBr₃ to a liquor inoculated with a culture from a ruminant animal foregut. FIG. 11 comprises a graph 1101 of the measured temperature on the upper y-axis 1102 shown in degrees Celsius (° C.), the measured CH₄ on the middle y-axis 1103 in percentage (%), and the measured dH₂ on the lower y-axis 1104 in milli Volts (mV) versus time on the x-axis 1105 measured in hours.

The plot 1005 of temperature is the temperature in the liquor recorded by a temperature sensor located in about the mid-point vessel and contacting the liquor as described in FIG. 7 700 and the accompanying text.

The temperature was monitored and the heat generated by the culture could be distinguished from the thermal energy normally applied to liquor by the heating elements of the culturing device to maintain the temperature of the liquor at the set point of 39° C.

The plot 1106 of CH₄ is the output of the SGX methane sensor as described in FIG. 8 800 and the accompanying text.

The plot 1006 of dH₂ is the voltage generated by a hydrogen fuel cell as described in FIG. 7 700 and the accompanying text.

The culturing device vessel was inoculated with a culture sourced from a freshly slaughtered grass-fed ewe hogget sheep of mixed breed. After resection of the reticulorumen organ, 2.5 L the rumen content was determined to have a pH Of 6.0 and then transferred to a 5 L jug and mixed with 2.5 L of rainwater. A 0.7 L aliquot of this mixture was used to inoculate the vessel of a culturing device containing 0.8 L of rainwater pre-heated to the culturing device setpoint temperature of about 39° C.

After inoculation, the culturing device was prescribed the movement algorithm described in FIG. 10 1000 above.

It will be appreciated that the time domains for the movement, typically seconds, are substantially smaller than the time domains culturing, typically days.

At the top of the graph 1101 are bars 1108 to 1114, indicating daily intervals delineated by abrupt downward pointing transients in the temperature plot 1105. Each bar 1108 to 1114 corresponds to a period of 24 hours with the temperature at the start of each period, except that the temperature transient for period 1108 is not shown. These temperature transients were caused by the daily feeding protocol which first extracts a volume of liquor from the vessel and then replaces that volume of liquor with an approximately equivalent volume of feed material at room temperature. The administration of the feed material at room temperature caused the abrupt change in temperature of the liquor until such time that the culturing device had caused the liquor temperature to rise to about the setpoint temperature.

The recording shown in the graph 1101 shows seven consecutive days of elapsed time, which started two complete days (48 hours) after inoculation. Each consecutive day is delineated by a single feed event and its consequential downward pointing temperature transient.

As described herein, the vessel of the present examples has an internal volume of about 2.0 L and is typically operated with 1.5 L of liquor and 0.5 L of headspace. A feeding event starts with a decanting of liquor at a prescribed angle as described in FIG. 5 and the accompanying text. In the present example the vessel was moved to 71 degrees to extract liquor material by decanting and to retain approximately two thirds (1 L) of the liquor in the vessel. The extracted volume of liquor, typically 0.5 L or less can be subjected to asynchronous analyses and/or discarded.

It will be appreciated that any fraction of liquor may be retained or extracted by prescribing the angle of movement to decant and extract liquor. In the present example, a hydraulic retention time for feed in the vessel was set to about 3 days by the daily extraction of vessel liquor of about ⅓rd of the liquor volume (0.5 L) and replacement with an equivalent volume of feed materials (0.5 L) during feeding. Other hydraulic retention times can be specified. For example, by decanting and replacing ¼ of the liquor daily specifies a hydraulic retention time of 4 days.

It will be appreciated that the volume of decanted liquor may be variable and/or reduced due to the conversion of liquor materials to volatile and gaseous substances thereby decreasing the liquor volume. However, by precisely decanting the liquor to a prescribed angle of movement permits retention of a known and more precise volume of liquor.

Typical analyses of the extracted liquor include for example asynchronous measurements including for example the determination of the mass, volume, pH, Eh, dry matter (DM), and VFA content.

Following the extraction of liquor, a replacement material comprising feed and buffer was administered to the vessel at a prescribed angle, as described in FIG. 4 400 and the accompanying text. In the present example the vessel was moved to −25 degrees which assists in the administration method.

The administration of feed used in the present example comprised weighing a mass 12 g of dried and cut forage plant lucerne, adding to the cut forage plant a mass of 475 g of artificial saliva, a bicarbonate-based buffer comprised of rainwater, Na₂HPO₄ (26 mM), NaHCO₃ (117.0 mM), NaCl (8.0 mM), KCl (8.0 mM), MgCl₂ (0.3 mM), and CaCl₂) (0.2 mM). The cut forage plant and the buffer were mixed to form a slurry with a volume of about 0.5 L. Any other material to be administered to the culture was added to the forage prior to the buffer and the volume adjusted and mixed.

The slurry was then administered to the vessel and then the culturing device was prescribed the movement algorithm described in FIG. 10 1000 above.

In the experiment CHBr₃ together with 1 mL of canola oil and 1 g of ground oats was added to the slurry to give a final CHBr₃ concentration of 6 μM in the 1.5 L of liquor and administered to the culturing device during the daily feeding protocol 1109, 1110, 1111, and 1112.

From the plot 1106 of CH₄ the effect of the added CHBr₃, a known potent inhibitor of methanogenesis, appears to be cumulative as can be seen with decreasing CH₄ up to an including the final day of CHBr₃ administration 1112. The recovery of methanogenesis after CHBr₃ administration was ceased when no CHBr₃ was administered with feed on 1113 and 1114, was rapid as shown in the plot 1106 of CH₄. The results from the plot 1106 are mirrored in the plot 1107 of the dH₂ which is anticipated to increase due to the reduction of methanogenesis and decrease when methanogenesis is increased.

Analysis of the extracted liquor using a calibrated pH meter showed that the culturing device maintained stable pH: 1108, pH 6.4; 1109, pH 6.2; 1110, pH 6.0; 1111, pH 6.2; 1112, pH 6.3; 1113, pH 6.4; 1114, pH 6.5 and near to pH 6.0 of the rumen from which the inoculation was sourced.

FIG. 12 shows an example recording 1200 of the effect on headspace pressure due to prescribed movement of the vessel and the emission of fermentation gases from a culture. The recording comprises a graph 1201 of measured pressure on the upper y-axis 1202 in Pascals and the prescribed movement and on the lower y-axis 1203 in degrees, versus time on the x-axis 1204 in seconds. The plot 1205 of pressure is the pressure on the headspace recorded by an electronic pressure sensor. The plot 1206 shown in degrees is the angle of the vessel containing liquor moved about an axis of rotation as explained by FIG. 9 and the accompanying description and comprises four cycles of prescribed movement of the vessel between −24 degrees and 44 degrees.

As can be seen from the graph 1201, the movement of the vessel causes the pressure inside the vessel containing the liquor and the headspace to change as reflected in the plot 1205 of the measured pressure. This indicates that moving of the vessel causes movement and thus mixing of the liquor and headspace fluids of the culture.

Also shown in the plot of the pressure 1205, are short lived (approximately 1 s) downward pointing transients 1207 that correspond to pulses of fermentation gases released from the culture and passed through a fermentation airlock.

It will be appreciated that such pulses 1207 can be enumerated and related to the volume of emitted gas after appropriate calibration of the pulse volume. It will further be appreciated that the enumeration of pulses 1207 as a function of time will yield the flow rate of emitted gases in real time.

Furthermore, by measuring the concentration of fermentation gases in the gas line prior to the fermentation airlock, for example CH₄ in as depicted in FIGS. 8 and 11 and the accompanying description, enables the determination volume and rate of gas emission.

### Example 3: Comparative Measurements Between Culturing Device and an Animal

The average sheep live weight is 60 kg and consumes 2 to 3% of its live weight in dry matter (DM) daily to produce 21 to 22 g of CH₄ per kg of DM eaten.

For an average sheep consuming 2 to 3%×60 kg=1.2 to 1.8 kg DM per day and yields 25 to 40 g of CH₄ per day.

The volume of CH₄ per day from 25 g and 40 g of CH₄ at 22° C. and atmospheric pressure is calculated using the ideal gas law: PV=nRT, where n is the number of moles of gas, R=8.314 J/mol K (ideal gas constant), P is the gas pressure=101325 Pa, and T is the temperature=295.15 K.

Using the ideal gas law, the average sheep produces 37.7 to 60.4 L CH₄ per day, which comprises foregut (95%) and hindgut CH₄ production (5%).

Consequently, the average sheep foregut produces 35.82 to 57.38 L CH₄ per day.

A 2 L vessel according to the present invention inoculated with rumen culture from a sheep was administered 15 g per day of dry lucerne, which contained 0.8 g DM per g of lucerne corresponding to 12 g DM per day and produces 0.5 to 1.0 L of gas per day estimated from a total gas capture.

The CH₄ level sits on average at about 18% according to convergent CH₄ sensors on several instruments run in parallel, which implied a yield of 0.09 to 0.18 L of CH₄/day.

Comparing the sheep foregut CH₄ production with the culturing device of the present invention requires an appropriate vessel volume and feed correction.

The average sheep has a rumen of volume of about 6 L and so the 2 L vessel volume requires a 2/6 volume correction applied to the sheep foregut CH₄ production.

A sheep with a live weight of 60 kg that is fed 2-3% DM per day consumes about 1.5 kg DM per day.

Administration to the culturing device with a 2 L vessel as described herein with 12 g DM per day requires a 12/1500 feed correction applied to the sheep foregut CH₄ production.

Applying the volume and feed corrections to the sheep foregut CH₄ per day yields:

35.82 to 57.38 L CH₄/per day×2/6×12/1500=0.10 to 0.15 L of CH₄ per day,

The corrected values for CH₄ per day for an average sheep foregut compared favourably to the 2 L vessel yield of 0.09 to 0.18 L of CH₄ per day and supports the utility of the present disclosure as a model of ruminant foregut CH₄ production.

### Example 4: Advantages of Culturing Devices

Amongst the advantages already described above, disclosed herein, are culturing devices that can culture microorganisms from the animal gut. The devices are highly configurable in terms of their modes of configuration, programmability of the movement means, and amenability to machine automation. Furthermore, the culturing devices disclosed herein are comparatively inexpensive to manufacture and operate to enable their widespread availability.

Some examples of the culturing devices, may be used to discover, develop, and assess feeds, treatments, additives, supplements, and devices, to improve the health and productivity and/or reduce methane emissions from farmed animals.

The culturing devices disclosed herein simplify the taking of measurements and the extraction of analytical samples, the administration of test materials, have low variance due to their automation and permit the cost-effective scale of testing required to achieve statistical significance.

The use of a culturing device may be used to develop treatments, feeds, and supplements to reduce methane gas emissions from farmed animals.

While the present disclosure has been illustrated by the description of the examples thereof, and while the examples have been described in detail, it is not the intention of the Applicant to restrict or in any way limit the scope of the appended claims to such detail. Additional advantages and modifications will readily appear to those skilled in the art. Therefore, the disclosure in its broader aspects is not limited to the specific details, representative apparatus and method, and illustrative examples shown and described. Accordingly, departures may be made from such details without departure from the spirit or scope of the of the general inventive concept.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A culturing device comprising:
(i) at least one vessel for containing a culture;
(ii) at least one sensor;
(iii) at least one conduit fluidically connected to said vessel providing a path for said culture;
(iv) a pressure adjustment means; and
(v) a control module,
wherein the at least one sensor is configured to detect at least one physical or chemical property of the culture,
and wherein said control module is configured to move said vessel and thereby cause at least a portion of the liquid phase of said culture to move between said vessel and said conduit and change at least one parameter of said culture.

2. The culturing device according to claim 1, wherein the sensor comprises a dissolved hydrogen sensor (dH₂) and the dH₂ sensor is configured to measure an amount or concentration of dissolved hydrogen in the culture.

3. The culturing device according to claim 1 or claim 2, which further comprises a volatile fatty acid adsorption means.

4. The culturing device according to any one of claims 1 to 3, further comprising a temperature adjustment means.

5. The culturing device according to any one of claims 1 to 4, wherein the at least one parameter of said culture is a change in the volatile fatty acid (VFA) concentration of said culture.

6. The culturing device according to any one of claims 1 to 5, wherein the at least one parameter of said culture is a change in pressure-.

7. The culturing device according to any one of claims 1 to 6, wherein the control module further comprises at least one instruction responsive module and at least one operation means, wherein the instruction responsive module activates the operation means to perform a task resulting in a change in a parameter associated with the culture or the culturing device.

8. The culturing device according to any one of claims 1 to 7, wherein control module is configured to move the vessel in a way which simulates the movement reflected in a gut organ derived from a ruminant animal.

9. The culturing device according to any one of claims 1 to 8, wherein the pressure adjustment means is configured to generate fluctuations in pressure that would occur naturally in a gut organ derived from a ruminant animal.

10. The culturing device according to any one of claims 1 to 9, wherein the control module is configured to receive an input signal or to produce an output signal.

11. The culturing device according to claim 10, wherein the input signal received by the control module causes the control module to change at least one parameter of the culture or the culturing device.

12. The culturing device according to claim 10 or claim 11, wherein the output signal produced by the sensor is stored on a storage medium.

13. The culturing device according to any one of claims 1 to 12 wherein the culture is selected from the content, extraction and excretion of a gut organ of a living or dead animal from the suborder Ruminantia or Tylopoda.

14. A method for evaluating the effect on a culture of any one or more of:
(i) a feed;
(ii) a feed supplement;
(iii) an intervention;
(iv) a medicament;
(v) microorganism;
(vi) a microorganism inhibitor;
(vii) a microorganism enhancer;
(viii) a methanogen inhibitor;
(ix) a methanogenesis inhibitor
the method comprising the steps of:
(a) inoculating a culturing device according to any one of claims 1 to 13 with a culture;
(b) introducing any one or more of (i) to (ix) to the culture;
(c) measuring at least one parameter associated with the culture from the at least one sensor,
thereby evaluating the effect on the culture of any one or more of (i) to (ix).

15. The method according to claim 14, wherein:
(i) the feed is selected from a plant, a leaf, a stem, a grain, a seed, a husk, a kernel, a pasture, a grass, a Gramineae, a legume, a Leguminosae, a clover, a lucerne, a fodder, a forage, a brassica, a herb, a chicory, a plantain, an algae, a macroalgae, a barley, a corn, a maize, a wheat, an oat, an amino acid, a peptide, a protein, a carbohydrate, a cellulose, a hemicellulose, a starch, a fat and an oil;
(ii) the feed is selected from a cut feed, a chopped feed, a ground feed, a preserved feed, a straw feed, a hay feed, a silage, baleage, a frozen feed, a chilled feed, a dried feed, a freeze dried feed, a rolled feed, a crushed feed, a ground feed, a pelleted feed, a tableted feed and a cooked feed;
(iii) the feed is selected from a digested feed, a pre-digested feed, a treated feed, a pre-treated feed, an enzyme-treated feed, an acid-treated feed and an incubated feed;
(iv) the feed supplement is selected from a vitamin, a drug, a chemical, an element, a trace element, a probiotic, a prebiotic, a postbiotic, a plant, an extract, an algae, a mineral, a trace element, a top dressing, a fluid, a solid, a lozenge, a capsule and a bolus;
(v) the medicament is selected from an agent, an adjuvant, a drug, a chemical, a biochemical, a biomolecule, a nucleic acid, an amino acid, a peptide, a protein, a pharmaceutical, a nutraceutical, a therapeutic, an antibiotic, an anthelminthic, an ionophore, an antiprotozoal, a defaunating agent, a fungicide, a fluid, a solid, a lozenge, a bolus, an electronic bolus and a device;
(vi) the microorganism is selected from a probiotic, a bacterium, an archaea, a fungi, a protozoa, a parasite, a helminth, a nematode, a cestode, a trematode, a virus, a phage, a spore, an egg and an oocyte
(vii) the microorganism inhibitor is selected from a drug, a chemical, an agent, an antibiotic, an antiprotozoal, a defaunation agent, an ionophore, an anthelminthic, an antifungal, a fungicide, a fluid, a solid, a lozenge, a bolus, an electronic bolus and a device;
(viii) the microorganism enhancer is selected from a carbohydrate, a sugar, glucose, glycerol, an oil, a vegetable oil, canola oil, olive oil, mineral oil, paraffin oil, a fat, a lipid, a liposome and a triacyl glyceride;
(ix) the methanogen or methanogenesis inhibitor is selected from a chemical, a compound, a drug, a gas, a liquid, an encapsulation, a liposome, a lipid nanoparticle, oxygen (O₂), ozone (O₃), peroxide, calcium peroxide, an antibiotic, an organohalogen, a haloform, chloroform, bromoform, iodoform, a seaweed, Asparagopsis sp., Asparagopsis extract, Asparagopsis matter, an alliin, an iso-alliin, an allicin, Mootral™, garlic extract, garlic matter, 3-Nitrooxypropanol (3-NOP), Bovaer™, a fluid, a solid, a lozenge, a bolus, a device, an electronic bolus, light, biocidal light, flavin, Co-factor F420, photobleaching agent, 420 nm light.
